SEQUENCE LISTING (86 sequences)

SEQ ID NO: 1 - DNA, 20 bp, Artificial Sequence
  Padlock Probe
acgggtggaagatggatgat

SEQ ID NO: 2 - DNA, 50 bp, Artificial Sequence
  Padlock Probe
agatcggaagagcgtcgtgtagggaaagctgagcaaatgttatcgaggtc

SEQ ID NO: 3 - DNA, 24 bp, Artificial Sequence
  Padlock Probe
gatcgtccttacacactagccgtc

SEQ ID NO: 4 - DNA, 20 bp, Artificial Sequence
  PCR Primer
acgggtggaagatggatgan

SEQ ID NO: 5 - DNA, 20 bp, Artificial Sequence
  PCR Primer
gacggctagtgtgtaaggac

SEQ ID NO: 6 - DNA, 23 bp, Artificial Sequence
  PCR Primer
ggctagtgtgtaaggacgatcnn

SEQ ID NO: 7 - DNA, 46 bp, Artificial Sequence
  Amplification Primer
caagcagaagacggcatacgagctctgagcaaatgttatcgaggtc

SEQ ID NO: 8 - DNA, 43 bp, Artificial Sequence
  Amplification Primer
aatgatacggcgaccaccgacactctttccctacacgacgctc

SEQ ID NO: 9 - DNA, 28 bp, Artificial Sequence
  PCR Primer
ttaaaggattttaggaattttattagtt

SEQ ID NO: 10 - DNA, 27 bp, Artificial Sequence
  PCR Primer
aaatactatcaaaaactacttccaaac

SEQ ID NO: 11 - DNA, 22 bp, Artificial Sequence
  PCR Primer
gttgtggttagatttggttttt

SEQ ID NO: 12 - DNA, 25 bp, Artificial Sequence
  PCR Primer
accttaacctccctaaaactaataa

SEQ ID NO: 13 - DNA, 23 bp, Artificial Sequence
  PCR Primer
aagtttttttagtaaggttggga

SEQ ID NO: 14 - DNA, 25 bp, Artificial Sequence
  PCR Primer
cactacactctatcctaaacaacaa

SEQ ID NO: 15 - DNA, 25 bp, Artificial Sequence
  PCR Primer
attttttggttttaggtttatagtg

SEQ ID NO: 16 - DNA, 24 bp, Artificial Sequence
  PCR Primer
aaatctctctcaaaaattccttaa

SEQ ID NO: 17 - DNA, 25 bp, Artificial Sequence
  PCR Primer
ttaatttggtttgttgattttagtt

SEQ ID NO: 18 - DNA, 25 bp, Artificial Sequence
  PCR Primer
ctcacctaaaaaatatataaatccc

SEQ ID NO: 19 - DNA, 25 bp, Artificial Sequence
  PCR Primer
gtgaataggttaagtgaggtagaag

SEQ ID NO: 20 - DNA, 25 bp, Artificial Sequence
  PCR Primer
aaaaaaatcaaacaccaactataaa

SEQ ID NO: 21 - DNA, 25 bp, Artificial Sequence
  PCR Primer
gggtgagtagtaggtttgtagtaaa

SEQ ID NO: 22 - DNA, 25 bp, Artificial Sequence
  PCR Primer
caaataacaccataaactaaaacaa

SEQ ID NO: 23 - DNA, 25 bp, Artificial Sequence
  PCR Primer
tttgttttaagtttttaaagggtaa

SEQ ID NO: 24 - DNA, 27 bp, Artificial Sequence
  PCR Primer
aaatactctaaatttctcacaacctac

SEQ ID NO: 25 - DNA, 26 bp, Artificial Sequence
  PCR Primer
gtaggttttaggaattttaggataga

SEQ ID NO: 26 - DNA, 25 bp, Artificial Sequence
  PCR Primer
taaaaccctttacatttcaataaat

SEQ ID NO: 27 - DNA, 28 bp, Artificial Sequence
  PCR Primer
ttttatttagagttgttttatgttaagg

SEQ ID NO: 28 - DNA, 25 bp, Artificial Sequence
  PCR Primer
atctcctataaatccccaattaata

SEQ ID NO: 29 - DNA, 22 bp, Artificial Sequence
  PCR Primer
gttttggtagagatttgtttgg

SEQ ID NO: 30 - DNA, 25 bp, Artificial Sequence
  PCR Primer
aaaaaaaacccctactctactactc

SEQ ID NO: 31 - DNA, 25 bp, Artificial Sequence
  PCR Primer
aggtgatatgaggaagtattgttat

SEQ ID NO: 32 - DNA, 26 bp, Artificial Sequence
  PCR Primer
aaacctccatactaaaaaatttacat

SEQ ID NO: 33 - DNA, 25 bp, Artificial Sequence
  PCR Primer
ttagattttattttggattttgaaa

SEQ ID NO: 34 - DNA, 25 bp, Artificial Sequence
  PCR Primer
ctctacaaaaacttaacccttaaaa

SEQ ID NO: 35 - DNA, 25 bp, Artificial Sequence
  PCR Primer
gaaaatttgattttaaaagaatgtg

SEQ ID NO: 36 - DNA, 25 bp, Artificial Sequence
  PCR Primer
ttttaaaaataacaaaatcaactcc

SEQ ID NO: 37 - DNA, 28 bp, Artificial Sequence
  PCR Primer
ttaattgaagattaaatatttttgagat

SEQ ID NO: 38 - DNA, 25 bp, Artificial Sequence
  PCR Primer
ctttaaaatttccttttaaccaaat

SEQ ID NO: 39 - DNA, 27 bp, Artificial Sequence
  PCR Primer
ggagtttttaaggtttttatatttttt

SEQ ID NO: 40 - DNA, 24 bp, Artificial Sequence
  PCR Primer
ccaacacacaacttctaaaactaa

SEQ ID NO: 41 - DNA, 25 bp, Artificial Sequence
  PCR Primer
ttaggaggtgtttagatgattttag

SEQ ID NO: 42 - DNA, 23 bp, Artificial Sequence
  PCR Primer
cccaatatatacacaaccaaaac

SEQ ID NO: 43 - DNA, 25 bp, Artificial Sequence
  PCR Primer
atgtttgtgaaagtaggagtttatt

SEQ ID NO: 44 - DNA, 25 bp, Artificial Sequence
  PCR Primer
tactcttatcccttctccctaatat

SEQ ID NO: 45 - DNA, 25 bp, Artificial Sequence
  PCR Primer
gattgttagtattgtagagggtttg

SEQ ID NO: 46 - DNA, 30 bp, Artificial Sequence
  PCR Primer
aacttcaataatacattaaaataaaatttt

SEQ ID NO: 47 - DNA, 25 bp, Artificial Sequence
  PCR Primer
gatttttagttttgtagtgttgagg

SEQ ID NO: 48 - DNA, 30 bp, Artificial Sequence
  PCR Primer
ctaataaaatctaaattcaaaaacacttat

SEQ ID NO: 49 - DNA, 25 bp, Artificial Sequence
  PCR Primer
tttgtgttagttttgggtttaatat

SEQ ID NO: 50 - DNA, 25 bp, Artificial Sequence
  PCR Primer
caaccttcaataaaaacaaactatt

SEQ ID NO: 51 - DNA, 25 bp, Artificial Sequence
  PCR Primer
taagttaggtgttgggagttaatag

SEQ ID NO: 52 - DNA, 25 bp, Artificial Sequence
  PCR Primer
taaaatatccacctcaactaaaatc

SEQ ID NO: 53 - DNA, 25 bp, Artificial Sequence
  PCR Primer
tgattttattttgaaagtgaagttt

SEQ ID NO: 54 - DNA, 26 bp, Artificial Sequence
  PCR Primer
attttcacaaaaactataaaacacaa

SEQ ID NO: 55 - DNA, 25 bp, Artificial Sequence
  PCR Primer
gatttgtttgtttttttaaattttg

SEQ ID NO: 56 - DNA, 25 bp, Artificial Sequence
  PCR Primer
aaattaattccaattacaccaataa

SEQ ID NO: 57 - DNA, 26 bp, Artificial Sequence
  PCR Primer
aaaatattgggattataggtatgagt

SEQ ID NO: 58 - DNA, 25 bp, Artificial Sequence
  PCR Primer
aacttctaaactaaccaaaacaaaa

SEQ ID NO: 59 - DNA, 25 bp, Artificial Sequence
  PCR Primer
tgttttaggaggtgaataaattaat

SEQ ID NO: 60 - DNA, 25 bp, Artificial Sequence
  PCR Primer
aaccttataaacttcacaatcaaac

SEQ ID NO: 61 - DNA, 30 bp, Artificial Sequence
  PCR Primer
tttatttaatatatgttggatgaataatta

SEQ ID NO: 62 - DNA, 23 bp, Artificial Sequence
  PCR Primer
ctaaaaccctcctcaataacttc

SEQ ID NO: 63 - DNA, 27 bp, Artificial Sequence
  PCR Primer
tgttaatgaatataatgttttgttttt

SEQ ID NO: 64 - DNA, 25 bp, Artificial Sequence
  PCR Primer
taatacccaactaactccctactaa

SEQ ID NO: 65 - DNA, 27 bp, Artificial Sequence
  PCR Primer
ttatagtttgggtgatagagtaagatt

SEQ ID NO: 66 - DNA, 27 bp, Artificial Sequence
  PCR Primer
aaaccctaaacaaaatactcaatataa

SEQ ID NO: 67 - DNA, 25 bp, Artificial Sequence
  PCR Primer
ggtagatatgttgttgtgtgtagaa

SEQ ID NO: 68 - DNA, 24 bp, Artificial Sequence
  PCR Primer
aaaaaaacttcataaccaaaactc

SEQ ID NO: 69 - DNA, 25 bp, Artificial Sequence
  PCR Primer
tatgatagaggtggtagtagaggtg

SEQ ID NO: 70 - DNA, 25 bp, Artificial Sequence
  PCR Primer
ttccaattatctcctaaacaaaata

SEQ ID NO: 71 - DNA, 27 bp, Artificial Sequence
  PCR Primer
aaaagtttagtatattttgtggttttt

SEQ ID NO: 72 - DNA, 30 bp, Artificial Sequence
  PCR Primer
caccaatatattataaaaaaactctttatt

SEQ ID NO: 73 - DNA, 25 bp, Artificial Sequence
  PCR Primer
ggggtagatattaggttttaaagag

SEQ ID NO: 74 - DNA, 23 bp, Artificial Sequence
  PCR Primer
aactacaaaaactcctcaacaaa

SEQ ID NO: 75 - DNA, 32 bp, Artificial Sequence
  Sequencing Primer
cactctttccctacacgacgctcttccgatct

SEQ ID NO: 76 - DNA, 58 bp, Artificial Sequence
  Adapter Oligonucleotide
caagcagaagacggcatacgaagagtctctatatgcatcgatgcagatcacgatccga

SEQ ID NO: 77 - DNA, 60 bp, Artificial Sequence
  Adapter Oligonucleotide
cgtcggatcgtgatctgcatcgatgcatatagagactcttcgtatgccgtcttctgcttg

SEQ ID NO: 78 - DNA, 60 bp, Artificial Sequence
  Adapter Oligonucleotide
aatgatacggcgaccaccgagatctacactctttccctacacgacgctcttccgatctnn

SEQ ID NO: 79 - DNA, 58 bp, Artificial Sequence
  Adapter Oligonucleotide
agatcggaagagcgtcgtgtagggaaagagtgtagatctcggtggtcgccgtatcatt

SEQ ID NO: 80 - DNA, 21 bp, Artificial Sequence
  PCR Primer
caagcagaagacggcatacga

SEQ ID NO: 81 - DNA, 13 bp, Artificial Sequence
  PCR Primer
agcggcatagtaa

SEQ ID NO: 82 - DNA, 33 bp, Artificial Sequence
  Sequencing Primer
tctagccttctcgcagcacatccctttctcaca

SEQ ID NO: 83 - DNA, 137 bp, Artificial Sequence
  Library Oligonucleotde
caagcagaagacggcatacgaagagtctctatatgcatcgatgcagatcacgatccgacggnnnnnnnnnnnnnnnnnnagatcggaagagcgtcgtgtagggaaagagtgtagatctcggtggtcgccgtatcatt

SEQ ID NO: 84 - DNA, 137 bp, Artificial Sequence
  Library Oligonucleotide
aatgatacggcgaccaccgagatctacactctttccctacacgacgctcttccgatctnnnnnnnnnnnnnnnnnnccgtcggatcgtgatctgcatcgatgcatatagagactcttcgtatgccgtcttctgcttg

SEQ ID NO: 85 - DNA, 21 bp, Artificial Sequence
  PCR Primer
caagcagaagacggcatacga

SEQ ID NO: 86 - DNA, 21 bp, Artificial Sequence
  PCR Primer
aatgatacggcgaccaccgag

(19) United States
(12) Patent Application Publication
(10) Pub. No.: US 2010/0273164 A1
(43) Pub. Date: Oct. 28, 2010

(54) Targeted and Whole-Genome Technologies to Profile DNA Cytosine Methylation

(75) Inventors: George M. Church, Brookline, MA (US); Madeleine Price Ball, Brookline, MA (US); Jin Li, Medford, MA (US)

Correspondence Address: BANNER & WITCOFF, LTD., 28 STATE STREET, SUITE 1800, BOSTON, MA, 02109-1701, US

(73) Assignee: President and Fellows of Harvard College, Cambridge, MA (US)

(21) Appl. No.: 12/726,411

(22) Filed: Mar. 18, 2010

Related U.S. Application Data
(60) Provisional application No. 61/162,913, filed on Mar. 24, 2009.

(51) Int. Cl. C12Q 1/68

(52) U.S. Cl. 435/6

(57) ABSTRACT

Methods and compositions for determining a methylated cytosine profile of a target nucleic acid sequence are provided.

### RELATED APPLICATIONS

This application claims priority to U.S. Provisional Patent Application No. 61/162,913, filed on May 24, 2009 and is hereby incorporated herein by reference in its entirety for all purposes.

### STATEMENT OF GOVERNMENT INTERESTS

This invention was made with government support under HG003170 awarded by the National Institutes of Health. The government has certain rights in the invention.

### BACKGROUND

1. Field of the Invention

Embodiments of the present invention relate in general to methods and compositions for profiling the methylation state of cytosine residues in a nucleic acid sample.

2. Description of Related Art

Cytosine methylation, an epigenetic modification of DNA, plays an important role in embryogenesis, cancer, and other human diseases (Goll, M. G. & Bestor, T. H., Eukaryotic cytosine methyltransferases. Annu Rev Biochem 74, 481-514 (2005); Suzuki, M. M. & Bird, A., DNA methylation landscapes: provocative insights from epigenomics. Nat Rev Genet 9 (6), 465-476 (2008); Feinberg, A. P. & Tycko, B., The history of cancer epigenetics. Nat Rev Cancer 4 (2), 143-153 (2004); Jiang, Y. H., Bressler, J., & Beaudet, A. L., Epigenetics and human disease. Annu Rev Genomics Hum Genet 5, 479-510 (2004)). Although a variety of methods are available to study cytosine methylation, many are limited by insufficient throughput, low accuracy, or inherent biases (Suzuki, M. M. & Bird, A., DNA methylation landscapes: provocative insights from epigenomics. Nat Rev Genet 9 (6), 465-476 (2008); Beck, S. & Rakyan, V. K., The methylome: approaches for global DNA methylation profiling. Trends Genet 24 (5), 231-237 (2008); Zilberman, D. & Henikoff, S., Genome-wide analysis of DNA methylation patterns. Development 134 (22), 3959-3965 (2007)).

### SUMMARY

Accordingly, the present invention is directed in part on the discovery of two new, complementary techniques for cytosine methylation profiling, bisulfite padlock probes (BSPPs), and methyl sensitive cut counting (MSCC), both of which utilize the power of next generation sequencing technology. In the first method, a set of ˜10,000 BSPPs complementary to target DNA (e.g., ENCODE regions) was designed (Birney, E. et al., Identification and analysis of functional elements in 1% of the human genome by the ENCODE pilot project. Nature 447 (7146), 799-816 (2007)). A pattern of low promoter methylation coupled with high gene body methylation was observed in highly expressed genes. Using the second method, MSCC, genome-wide data was gathered for 1.4 million HpaII sites and it was determined that gene body methylation in highly expressed genes was a consistent phenomenon over the entire genome. In addition, it was determined that expression-related differences in promoter methylation were larger outside of CpG islands than within, highlighting the usefulness of DNA methylation profiling technologies like BSPP and MSCC that are not strongly biased in favor of CpG islands.

Accordingly, a method for determining a methylated cytosine profile of a target nucleic acid sequence is provided. In certain exemplary embodiments, the method includes the steps of providing a sample of nucleic acid sequences, contacting the sample with a chemical agent (e.g., bisulfite) to convert unmethylated cytosine residues in the nucleic acid sequences to uracil residues, contacting the sample with a plurality of nucleic acid probes, wherein the probes are designed to hybridize randomly along a target nucleic acid sequence, allowing hybridization of the plurality of nucleic acid probes to the target nucleic acid sequence, forming a plurality of circular nucleic acid sequences, each of the circular sequences comprising a nucleic acid probe sequence and a target nucleic acid sequence, amplifying the plurality of circular nucleic acid sequences to form a plurality of amplified target nucleic acid sequences, and sequencing the amplified target nucleic acid sequences. In certain aspects, probes (e.g., padlock probes) are designed to hybridize to promoter regions along a target nucleic acid sequence. In other aspects, amplification primers hybridize to nucleic acid probe sequences during the step of amplifying. In still other aspects, the target nucleic acid sequence is any combination of genomic DNA (e.g., whole genome DNA), one or more genes, and one or more promoter regions.

In other exemplary embodiments, the method includes the steps of providing a sample of nucleic acid sequences, cleaving the nucleic acid sequences in a methylation-dependent manner to generate a plurality of cleaved target nucleic acid sequences, ligating first adapter sequence tags to the 5′ ends of cleaved target nucleic acid sequences and second adapter sequence tags to the 3′ ends of the cleaved target nucleic acid sequences, amplifying the cleaved target nucleic acid sequences having first and second adapter sequence tags ligated thereto, and sequencing the amplified, cleaved target nucleic acid sequences. In certain aspects, the step of cleaving the nucleic acid sequences in a methylation-dependent manner comprises contacting the nucleic acid sequences with a methyl sensitive restriction enzyme to cleave unmethylated CpG dinucleotide sequences. In other aspects, amplification primers hybridize to the first or the second adapter sequence tags during the step of amplifying. In other aspects, the target nucleic acid sequence is any combination of genomic DNA (e.g., whole genome DNA), one or more genes, and one or more promoter regions. In certain aspects, the method further includes the step of comparing the methylated cytosine profile of the target nucleic acid sequence to a methylated cytosine profile of a control library, such as, e.g., a control library is generated by contacting a target nucleic acid sequence with a methylation-insensitive enzyme (e.g., MspI).

In other exemplary embodiments, a method for determining a complementary methylated cytosine library of a target nucleic acid sequence is provided. The method includes the steps of providing a sample of nucleic acid sequences, cleaving the nucleic acid sequences in a methylation-dependent manner to generate a plurality of cleaved target nucleic acid sequences, blocking the ends of the cleaved target nucleic acid sequences to prevent the cleaved target nucleic acid sequences from contributing to library construction, and contacting the blocked, cleaved target nucleic acid sequences with a methylation-insensitive enzyme to create a complementary methylated cytosine library that comprises a plurality of nucleic acid sequences that were not cleaved in a methylation-dependent manner. In certain aspects, the blocking step includes dephosphorylating the 5′ ends of the cleaved target nucleic acid sequences.

Further features and advantages of certain embodiments of the present invention will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIGS. 1A-1C depict bisulfite padlock probe (BSPP) technology enabling accurate measurement of methylation levels. (A) A BSPP experimental scheme. Two hybridizing locus-specific arms (blue) were connected by a 50 base pair common “backbone” sequence (green). Approximately 10,000 BSPPs were designed to target CpG sites in bisulfite-treated DNA with a CpG located at the 3′ end of the polymerized span (red). Circles were formed by addition of polymerase, dNTP, and ligase, and were subsequently amplified using the backbone sequence as primers. Sequencing was then performed using an Illumina Genome Analyzer with a primer matching the backbone sequence. 28 bases of arm sequence were read through before sequencing informative positions within the span (read lengths were 36 bases in total). (B) Correlation of methylation level in the technical replicates (Pearson coefficient r=0.965). (C) Correlation of BSPP methylation with the methylation levels determined by bisulfite PCR followed by Sanger sequencing at 33 locations (r=0.966).

FIGS. 2A-2F graphically depict methylation versus gene positions, split by gene expression level. (A) Running median methylation vs. gene position for high and low expression genes in ENCODE regions of the GM06990 cell line (based on BSPP data). (B)-(F) were based on methyl sensitive cut counting (MSCC) data and share the same key. (B) Running average methylation vs. gene position for all genes in the PGP1 EBV-transformed lymphoblastoid cell line, and split into five groups based on expression level. Contribution of each MSCC data point was normalized for local CpG density MspI control counts and, for sites within the gene, for gene length. (C) Running average methylation vs. position relative to transcription start site (TSS) (MSCC). The methylation pattern appeared to have two valleys on either side of TSS. This was very similar to the pattern of H3K4 methylation in promoter regions (Barski, A. et al., High-resolution profiling of histone methylations in the human genome. Cell 129 (4), 823-837 (2007)). (D) Running average methylation vs. position relative to transcriptional end of genes (for genes at least 15 kb in length). (E) Running average methylation vs. position relative to transcription start site for locations within CpG islands. (F) Running average methylation vs. position relative to transcription start site for locations outside CpG islands.

FIGS. 3A-3B depict MSCC technology allowing accurate estimate of methylation levels. (A) Scheme of generation of a methyl sensitive cut site library. (1) HpaII digestion cuts genomic DNA at all unmethylated CCGG sites only. (2) The first adapter containing an MmeI recognition site was ligated. (3) MmeI digestion cut into the unknown genomic sequence to produce an 18-19 base pair tag. (4) A second adapter was added by ligation. (5) The library was amplified and sequenced. The number of reads for a given site was correlated with the amount of digestion that occurred there and was thus an indication of methylation level. (B) BSPP methylation vs. MSCC counts data was grouped into 22 equal bins according to the BSPP-determined methylation levels. The average number of counts (black points) were linearly related to the average methylation of a bin (blue best fit line is shown). The standard deviation for each data point was also shown (horizontal green), along with the standard deviation for a Poisson distribution with the average value (horizontal blue).

FIGS. 4A-4B depict methylation profiles of individual genes. (A) Individual genes were plotted according to the average MSCC counts found in the promoters (horizontal axis, −400 to +1000 relative to start) and gene bodies (vertical axis, between the gene end and +2000 relative to start). The color of each point reflects the expression level of that gene. Only data points from outside CpG islands and only genes with at least 5 data points in each region were used. (B) A histogram of average gene body methylation appeared to be bimodally distributed (gray). When split into two equally sized groups based on gene expression, high expression genes (red) match the right peak and low expression genes (blue) matched the left peak. Only genes with at least 50 data points in the gene body region were used.

FIGS. 5A-5D depict BSPP capturing and correlation of probe observations for GM06990. (A) Padlock probes were expected to give rise to library molecules of 155 base pairs (arrow) after amplification. This band was purified and used for Sanger and Illumina Genome Analyzer sequencing. The high band (271 base pairs) was the result of amplification products produced by polymerization making an extra trip around the circularized molecule. The low band (approximately 45 base pairs) was derived from primers. M: 25 base pair DNA ladder (Invitrogen). 1 and 2: two technical replicates. The same patterns of DNA bands were observed for other samples (PGP1L, PGP9L, PGP9F, and PGP9 iPS). (B) A histogram of the number of reads each probe observed, probes were sorted according to the number of reads observed. (C) A comparison of the number of reads for individual probes between technical replicates was highly correlated (Pearson correlation r=0.956, Spearman ranked correlation ρ=0.968). (D) A histogram of the number of reads for each probe was observed for each run. Probes were sorted according to the number of reads observed.

FIGS. 6A-6H depict histograms of CpG methylation for BSPP data in GM06990 and PGP cell lines. (A) GM06990 EBV-transformed B-lymphocytes. (B) PGP1 EBV-transformed B-lymphocytes. (C) PGP9 EBV-transformed B-lymphocytes. (D) PGP9 fibroblasts. (E) PGP9 fibroblasts. (F) PGP1 induced pluripotent cells. (G) PGP9 induced pluripotent cells, clone 1. (H) PGP9 induced pluripotent cells, clone 2.

FIG. 7 depicts a histogram of correlations for methylation state of same-strand CpG pairs. For probes capturing more than a single CpG, the subset of sites for which both CpG's had intermediate methylation levels was taken (between 20% and 80% and at least 100 total reads), and the correlation of methylation state on individual strands for each pair of sites was determined. Sites were generally positively correlated with a coefficient of around 0.5. A mixture entirely of “C & C” and “T & T” haplotypes would be perfectly correlated, with a coefficient of 1; a mixture of “C & T” and “T & C” would be perfectly anti-correlated with a coefficient of −1; a random mixture would give rise to a coefficient of 0.

FIGS. 8A-8D depict correlations of BSPP methylation data with chromatin immunoprecipitation data. Using chromatin immunoprecipitation (ChIP) data from the ENCODE project produced for histone modifications in the GM06990 cell line, methylation measurements for individual CpGs were compared with the ChIP scores at those locations. It was determined that (A) methylation was positively correlated with H3K36me3, and that (B) methylation was negatively correlated with H3K27me3. These data are consistent with how these histone modifications are distributed in expressed versus inactive genes. (C) H3K36me3 was high in the gene body of highly expressed genes, and so it is positively correlated with the observation of high methylation in highly expressed genes. (D) H3K27me3 was high in the gene body of inactive genes, and so it was negatively correlated.

FIGS. 9A-9D depict a comparison of methylation levels at individual sites between PGP cell lines. Using the methylation levels gathered with bisulfite padlock probes, methylation at individual sites between cell lines was compared. (A) PGP1 EBV-transformed B-lymphocyte vs. PGP9 EBV-transformed B-lymphocyte (Pearson correlation r=0.85, Spearman ranked correlation ρ=0.87). (B) PGP9 EBV-transformed B-lymphocyte vs PGP9 fibroblast (Pearson correlation r=0.63, Spearman ranked correlation ρ=0.63). (C) PGP9 fibroblast vs. PGP9 induced pluripotent clone 1 (Pearson correlation r=0.46, Spearman ranked correlation ρ=0.45). (D) PGP9 fibroblast vs. PGP9 induced pluripotent clone 2 (Pearson correlation r=0.46, Spearman ranked correlation ρ=0.45).

FIGS. 10A-10G depict methylation versus position for PGP cell line using BSPP data. Running median methylation of high expression and low expression genes within the ENCODE regions of PGP cell lines, based on BSPP data. Although these cell lines had different amounts of genomic methylation (see FIG. 5 for histograms), there was a consistent pattern: high expression genes had a consistent pattern of low promoter methylation coupled with high gene body methylation; low expression genes had a constant methylation throughout that varied depending on the overall levels of methylation in the sample. All panels share the same key. (A) PGP1 lymphocyte. (B) PGP9 lymphocyte. (C) PGP1 fibroblast. (D) PGP9 fibroblast. (E) PGP1 induced pluripotent cells. (F) PGP9 induced pluripotent cells (clone 1). (G) PGP9 induced pluripotent cells (clone 2).

FIGS. 11A-11C depict histograms of the number of counts for MSCC data. (A) Histogram of number of sites for each MSCC HpaII counts value in each replicate. (B) Histogram of number of sites for each MSCC MspI control counts value. (C) Two dimensional histogram showing the correlation between counts from MSCC HpaII replicate 1 and replicate 2 (r=0.818).

FIGS. 12A-12B depict a prediction of methylation for individual MSCC locations for a given number of counts. Horizontal bars denote the median methylation for a given range of counts, boxes mark the 25th and 75th percentiles, whiskers mark the 5th and 95th percentiles. (A) Methylation levels for individual tags. (B) Methylation levels for combined tag counts. Some sites have data for both possible tags, these can be added together to create a more accurate methylation prediction.

FIG. 13 depicts promoter versus gene body for individual genes, using all data points (based on MSCC data). This figure is plotted the same way as FIG. 4, except that all data points (both inside and outside CpG islands) were used. Gene promoter methylation is the horizontal axis, gene body methylation is the vertical axis, and color reflects gene expression rank. Only genes with at least ten data points in each region were used.

FIGS. 14A-14C graphically depict the effects of CpG density and methylation in genes with different levels of expression. (A) High CpG promoters (65% of all promoters) tend to have little methylation regardless of expression. (B) Intermediate CpG promoters (16% of promoters) tend to have low levels of methylation in highly expressed genes and high levels of methylation in weakly expressed genes. (C) Low CpG promoters (28% of promoters) tend to be highly methylated regardless of gene expression.

FIGS. 15A-15B depict individual sites comparison of BSPP methylation vs. MSCC HpaII counts for single and paired tags. There were a total of 381 sites and, of those, 345 had MSCC data for both tags (“paired”) for a total of 726 tags. (A) The plot of individual tag counts vs. BSPP methylation for the 726 individual tags. (B) A plot of combined MSCC tag counts for the 345 sites with paired tags showed that the data became more accurate for these sites. The sum of paired tag counts (B) had a stronger correlation to methylation (B: r=−0.73, ρ=−0.79) than individual tags (A: r=−0.63, ρ=−0.70). Of the 1.4 million MSCC sites, most (888k, 63%) had paired tags.

FIGS. 16A-16D depict “inverse library” results. Preliminary results with an “inverse library” of tags derived from methylated CCGG sites. The library was constructed by dephosphorylating a HpaII digest, blocking them from ligation. The DNA was then cut at remaining CCGG sites with the methylation-insensitive isoschizomer MspI and a library was constructed from these ends as before. With an inverse library, absolute methylation estimates could be made in the following manner: Based on the estimated average of 1.7 inverse library counts per 100% methylated site (A) and the estimated average of 8.9 MSCC HpaII library counts per 0% methylated site (FIG. 3B), inverse library counts are normalized to HpaII counts by multiplying by 5.2. Then, for each site: Normalized sum counts=normalized inverse library counts+HpaII library counts. Then, using only sites with a normalized sum of at least 7, Estimated methylation=(normalized inverse library counts)/(normalized sum counts). (A) “Inverse library” single tag counts vs. methylation as determined by BSPP. These were positively correlated with methylation (r=0.30, ρ=0.31). When data was averaged in 20 bins as per FIG. 3B, a linear fit of f(x)=a*x to the average values found a value of a=0.58, indicating that tags from fully methylated sites produce an average of approximately 1.7 counts. (B) “Inverse library” combined tag counts for paired sites vs. BSPP methylation. As with the original MSCC library, these were more strongly correlated with methylation (r=0.36, ρ=0.38). (C) Estimated methylation based on combined HpaII and inverse library counts for single tags with a normalized counts sum of at least 7 (r=0.77, ρ=0.78). (D) Averaged estimated methylation for paired tag locations where both tags had a normalized counts sum of at least 7 (r=0.85, ρ=0.87).

FIG. 17 depicts estimates of increased MSCC accuracy with more sequencing reads. The probabilities over 70, 80 and 90% are highlighted in light green, yellow and red, respectively.

### DETAILED DESCRIPTION

The principles of the present invention may be applied with particular advantage in methods of detecting nucleic acid (e.g., DNA) methylation patterns and changes in methylation patterns in nucleic acid sequences such as e.g., one or more genes or a whole genome. In certain exemplary embodiments, methods and compositions to detect nucleic acid methylation relating to genome instability that leads to a disease state(s) or a change in general health are provided. As used herein, the terms “methylation biomarker,” “disease-specific methylated restriction site pattern” and “methylation fingerprint,” refer to any sequence of nucleotides, such as CpG rich regions, where the 5′ position of any cytosine base becomes methylated. These regions may be found in any nucleotide sequence including, but not limited to, promoters, regulatory elements, enhancers, and gene coding sequences. Changes in any methylation fingerprint may be an indicator of genome instability and may be useful in the diagnosis of disease. For example, changes in a methylation fingerprint may alter the accessibility of the DNA binding proteins to bind to the DNA.

As used herein, a “nucleic acid target region” refers to a nucleic acid sequence that is examined using the methods disclosed herein. A nucleic acid target region includes whole-genome DNA, a segment of genomic DNA (e.g., a gene, a promoter region and the like), whole mitochondrial DNA, a segment of mitochondrial DNA and the like. In the context of methods for phenotype identification, the invention provides methods for identifying the methylation state of a nucleic acid target gene region and/or the methylation state of a nucleotide locus, a nucleic acid target gene region can also refer to an amplified product of a nucleic acid target gene region, including an amplified product of a treated nucleic acid target gene region, where the nucleotide sequence of such an amplified product reflects the methylation state of the nucleic acid target gene region. One skilled in the art would recognize that the size or length of the nucleic acid target gene region may vary depending on the limitation, or limitations, of the equipment used to perform the analysis. The nucleic acid target gene region may comprise more than one gene of interest, at least one gene of interest, a portion of a gene of interest, a promoter of a gene of interest or any combination of these. Correspondingly, a sequential or non-sequential series of nucleic acid target gene regions may be analyzed and exploited to map an entire gene or genome. The intended target will be clear from the context or will be specified.

As used herein, the “methylation state” of a nucleic acid target nucleic acid sequence refers to the presence or absence of one or more methylated nucleotide bases or the ratio of methylated cytosine to unmethylated cytosine for a methylation site in a nucleic acid target region. For example, a nucleic acid target region containing at least one methylated cytosine is considered methylated (i.e., the methylation state of the nucleic acid target gene region is methylated). A nucleic acid target gene region that does not contain any methylated nucleotides is considered unmethylated. Similarly, the methylation state of a nucleotide locus in a nucleic acid target gene region refers to the presence or absence of a methylated nucleotide at a particular locus in the nucleic acid target gene region. For example, the methylation state of a cytosine at the 7th nucleotide in a nucleic acid target gene region is methylated when the nucleotide present at the 7th nucleotide in the nucleic acid target gene region is 5-methylcytosine. Similarly, the methylation state of a cytosine at the 7th nucleotide in a nucleic acid target gene region is unmethylated when the nucleotide present at the 7th nucleotide in the nucleic acid target gene region is cytosine (and not 5-methylcytosine). Correspondingly, the ratio of methylated cytosine to unmethylated cytosine for a methylation site or sites can provide a methylation state of a nucleic acid target region.

As used herein, a “characteristic methylation state” refers to a unique or specific data set comprising the location of at least one, a portion of the total or all of the methylation sites of a nucleic acid, a nucleic acid target gene region or a gene of a sample obtained from an organism, a tissue or a cell.

As used herein, “methylation ratio” refers to the number of instances in which a molecule or locus is methylated relative to the sum of methylated and unmethylated sites (i.e., in the entire sample). Methylation ratio can be used to describe a population of individuals or a sample from a single individual. For example, a methylation ratio at a single locus can be used to compare different nucleic acid molecules derived from one or more samples (e.g., cells, tissues and the like) of a single person, a specific person and at least one other person, or among a group of people. Methylation ratios can be used, for example, to describe the degree to which a nucleotide locus or nucleic acid region is methylated in a population of individuals. Thus, when methylation in a first population or pool of nucleic acid molecules is different from methylation in a second population or pool of nucleic acid molecules, the methylation ratio of the first population or pool will be different from the methylation ratio of the second population or pool. Such a ratio also can be used, for example, to describe the degree to which a nucleotide locus or nucleic acid region is methylated in a single individual. For example, such a ratio can be used to describe the degree to which a nucleic acid target gene region of a group of cells from a tissue sample are methylated or unmethylated at a nucleotide locus or methylation site.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine Typical bases for RNA are uracil, adenine, cytosine and guanine Correspondingly a “methylation site” is the location in the target gene nucleic acid region where methylation has occurred, or has the possibility of occurring. For example a location containing CpG is a methylation site wherein the cytosine may or may not be methylated.

As used herein, a “methylation site” refers to a nucleotide within a nucleic acid, nucleic acid target gene region or gene that is susceptible to methylation either by natural occurring events in vivo or by an event instituted to chemically methylate the nucleotide in vitro.

As used herein, the term “methylation sensitive enzyme” refers to an enzyme that cleaves in a methylation-dependent manner, i.e., the enzyme either preferentially cleaves methylated recognition sites or preferentially cleaves unmethylated recognition sites. An example of an enzyme that cleaves a methylated recognition site is BisI. Examples of enzymes that preferentially cleave unmethylated recognition sites include, but are not limited to, AatII, AciI, AclI, AgeI, AscI, AsiSI, AvaI, BceAI, BmgBI, BsaAI, BsaHI, BsiEI, BsiWI, BsmBI, BspDI, BsrFI, BssHII, BstBI, BstUI, BtgZI, EagI, FauI, FseI, FspI, HaeII, HgaI, HhaI, HinPII, HpaII, Hpy99I, HpyCH4IV, MluI, Nael, NarI, NgoMIV, NotI, NruI, PaeR7I, PmlI, PvuI, RsrII, SacIl, SalI, SfoI, SgrAI, SmaI, ZraI and the like.

The term “methylation-insensitive enzyme,” as used herein, refers to any enzyme that will cut a nucleic acid sequence at a CpG site with or without a 5′-methyl cytosine. In other words, a methylation insensitive enzyme will cleave a methylation restriction site independent of its methylation status. For example, one methylation insensitive enzyme is MspI.

In certain exemplary embodiments, the presence or absence of one or more methylated or unmethylated nucleotides may be identified as indicative of a disease state associated with methylated or unmethylated DNA, such as a neoplastic disease. In other embodiments, the presence or absence of one or more methylated or unmethylated nucleotides may be identified as indicative of a normal, healthy or disease free state. In still other embodiments, an abnormal ratio of methylated nucleic acid target gene molecules relative to unmethylated nucleic acid target gene molecules in a sample may be indicative of a disease state associated with methylated or unmethylated DNA, such as a neoplastic disease. For example, a relatively high number or a relatively low number of methylated nucleic acid target gene molecules compared to the relative amount in a normal individual may be indicative of a disease state associated with methylated or unmethylated DNA, such as a neoplastic disease. In other embodiments, an abnormal ratio of methylated nucleotide at a nucleotide locus relative to unmethylated nucleotide at a nucleotide locus in a nucleic acid target gene molecule can be indicative of a disease state associated with methylated or unmethylated DNA, such as a neoplastic disease. For example, a relatively high number or a relatively low number of methylated nucleotide loci compared to the relative amount in a normal individual can be indicative of a disease state associated with methylated or unmethylated DNA, such as a neoplastic disease.

Diseases associated with a modification of the methylation of one or more nucleotides include, for example: leukemia (Aoki E. et al., “Methylation status of the p151NK4B gene in hematopoietic progenitors and peripheral blood cells in myelodysplastic syndromes,” Leukemia 14(4):586-593 (2000); Nosaka, K. et al., “Increasing methylation of the CDKN2A gene is associated with the progression of adult T-cell leukemia” Cancer Res. 60(4):1043-1048 (2000); Asimakopoulos F A et al., “ABL 1 methylation is a distinct molecular event associated with clonal evolution of chronic myeloid leukemia” Blood 94(7):2452-2460 (1999); Fajkusova L. et al., “Detailed Mapping of Methylcytosine Positions at the CpG Island Surrounding the Pa Promoter at the bcr-abl Locus in CML Patients and in Two Cell Lines, K562 and BV173” Blood Cells Mol. Dis. 26(3):193-204 (2000); Litz C. E. et al., “Methylation status of the major breakpoint cluster region in Philadelphia chromosome negative leukemias” Leukemia 6(1):35-41 (1992)), head and neck cancer (Sanchez-Cespedes M. et al. “Gene promoter hypermethylation in tumors and serum of head and neck cancer patients” Cancer Res. 60(4):892-895 (2000)), Hodgkin's disease (Garcia J. F. et al. “Loss of p16 protein expression associated with methylation of the p161NK4A gene is a frequent finding in Hodgkin's disease” Lab Invest. 79(12):1453-1459 (1999)), gastric cancer (Yanagisawa Y. et al., “Methylation of the hMLH1 promoter in familial gastric cancer with microsatellite instability” Int. J. Cancer 85(1):50-53 (2000)), prostate cancer (Rennie P. S. et al., “Epigenetic mechanisms for progression of prostate cancer” Cancer Metastasis Rev. 17(4):401-409 (1998-99)), renal cancer (Clifford, S. C. et al., “Inactivation of the von Hippel-Lindau (VHL) tumor suppressor gene and allelic losses at chromosome arm 3p in primary renal cell carcinoma: evidence for a VHL-independent pathway in clear cell renal tumourigenesis” Genes Chromosomes Cancer 22(3):200-209 (1998), bladder cancer (Sardi, I. et al., “Molecular genetic alterations of c-myc oncogene in superficial and locally advanced bladder cancer” Eur. Urol. 33(4):424-430 (1998), breast cancer (Mancini, D. N. et al., “CpG methylation within the 5′ regulatory region of the BRCA1 gene is tumor specific and includes a putative CREB binding site” Oncogene 16(9):1161-1169 (1998); Zrihan-Licht S. et al., “DNA methylation status of the MUC1 gene coding for a breast-cancer-associated protein” Int. J. Cancer 62(3):245-251 (1995); Kass, D. H. et al., “Examination of DNA methylation of chromosomal hot spots associated with breast cancer,” Anticancer Res. 13(5A):1245-1251 (1993)), Burkitt's lymphoma (Tao, Q. et al., “Epstein-Barr virus (EBV) in endemic Burkitt's lymphoma: molecular analysis of primary tumor tissue” Blood 91(4):1371-1381 (1998)), Wilms tumor (Kleymenova, E. V. et al., “Identification of a tumor-specific methylation site in the Wilms tumor suppressor gene” Oncogene 16(6):713-720 (1998)), Prader-Willi/Angelman syndrome (Zeschnigh et al. “Imprinted segments in the human genome: different DNA methylation patterns in the Prader-Willi/Angelman syndrome region as determined by the genomic sequencing method” Human Mol. Genetics (6)3:387-395 (1997); Fang P. et al., “The spectrum of mutations in UBE3A causing Angelman syndrome” Human Mol. Genetics 80:129-135 (1999)), ICF syndrome (Tuck-Muller et al., “CMDNA hypomethylation and unusual chromosome instability in cell lines from ICF syndrome patients” Cytogenet Cell Genet. 89(1-2):121-128 (2000)), dermatofibroma (Chen, T. C. et al., “Dermatofibroma is a clonal proliferative disease” J. Cutan Pathol 27(1):36-39 (2000)), hypertension (Lee, S. D. et al., “Monoclonal endothelial cell proliferation is present in primary but not secondary pulmonary hypertension” J. Clin. Invest. 101(5):927-934 (1998)), pediatric neurological disorders (Campos-Castello, J. et al., “The phenomenon of genomic ‘imprinting’ and its implications in clinical neuropediatrics” Rev. Neurol. 28(1):69-73 (1999)), autism (Klauck, S. M. et al., “Molecular genetic analysis of the FMR-1 gene in a large collection of autistic patients” Hum Genet 100(2):224-229 (1997)), ulcerative colitis (Gloria, L. et al., “DNA hypomethylation and proliferative activity are increased in the rectal mucosa of patients with long-standing ulcerative colitis” Cancer 78(11):2300-2306 (1996)), fragile X syndrome (Hornstra, I. K. et al., “High resolution methylation analysis of the FMR1 gene trinucleotide repeat region in fragile X syndrome” Human Mol. Genetics 2(10):1659-1665 (1993)), and Huntington's disease (Ferluga, J. et al., “Possible organ and age-related epigenetic factors in Huntington's disease and colorectal carcinoma” Med. Hypotheses 29(1):51-54 (1998)).

Additional diseases associated with the epigenetic state of DNA include, but are not limited to, low grade astrocytoma, anaplastic astrocytoma, glioblastoma, medulloblastoma, colon cancer, lung cancer, pancreatic cancer, endometrial cancer, neuroblastoma, headaches, sexual malfunction, primary myxedema, pernicious anemia, Addison's disease, myasthenia gravis, juvenile diabetes, idiopathic thrombocytopenic purpura, multiple sclerosis, rheumatoid arthritis, scleroderma, and other disorders such as CNS malfunctions, damage or disease, symptoms of aggression or behavioral disturbances, clinical, psychological and social consequences of brain damage, psychotic disturbances and personality disorders, dementia and/or associated syndromes, cardiovascular disease, malfunction and damage, malfunction, damage or disease of the gastrointestinal tract, malfunction, damage or disease of the respiratory system, lesion, inflammation, infection, immunity and/or convalescence, malfunction, damage or disease of the body as an abnormality in the developmental process, malfunction, damage or disease of the skin, the muscles, the connective tissue or the bones, endocrine and metabolic malfunction, damage or disease, and also can be associated with undesired drug interactions.

Increased or decreased levels of methylation have been associated with a variety of diseases. Methylation or lack of methylation at defined positions can be associated with a disease or a disease-free state. The methods disclosed herein can be used with methods of determining the propensity of a subject to disease, diagnosing a disease, and determining a treatment regimen for a subject having a disease.

The methylation state of a variety of nucleotide loci and/or nucleic acid regions are known to be correlated with a disease, disease outcome, and success of treatment of a disease, and also may be used to distinguish disease types that are difficult to distinguish according to the symptoms, histologic samples or blood or serum samples. For example, CpG island methylator indicator phenotype (CIMP) is present in some types of ovarian carcinomas, but not in other types (Strathdee, et al., Am. J. Pathol. 158:1121-1127 (2001)). In another example, methylation may be used to distinguish between a carcinoid tumor and a pancreatic endocrine tumor, which may have different expected outcomes and disease treatment regimens (Chan et al., Oncogene 22:924-934 (2003)). In another example, H. pylori-dependent gastric mucosa associated lymphoid tissue (MALT) lymphomas are characterized as having several methylated nucleic acid regions, while those nucleic acid regions in H. pylori-independent MALT lymphomas are not methylated (Kaneko et al., Gut 52:641-646 (2003)). Similar relationships with disease, disease outcome and disease treatment have been correlated with hypomethylation or unmethylated nucleic acid regions or unmethylated nucleotide loci.

Methods related to the disease state of a subject may be performed by collecting a sample from a subject, treating the sample with a reagent that modifies a nucleic acid target sequence as a function of the methylation state of the nucleic acid target sequence, subjecting the sample to methylation specific amplification, then detecting one or more fragments that are associated with a disease or that are associated with a disease-free state. In certain embodiments, the fragments are detected by measuring the mass of the nucleic acid target gene molecule or nucleic acid target gene molecule fragments. Detection of a nucleic acid target gene sequence or nucleic acid target gene sequence fragment can identify the methylation state of a nucleic acid target gene molecule or the methylation state of one or more nucleotide loci of a nucleic acid target gene molecule. Identification of the methylation state of a nucleic acid target gene sequence or the methylation state of one or more nucleotide loci of a nucleic acid target gene sequence can indicate the propensity of the subject toward one or more diseases, the disease state of a subject, or an appropriate or inappropriate course of disease treatment or management for a subject.

There are many hybridization-based assays that comprise a hybridization step that forms a structure or complex with a target polynucleotide, such as a fragment of genomic DNA, and an enzymatic processing step in which one or more enzymes either recognize such structure or complex as a substrate or are prevented from recognizing a substrate because it is protected by such structure or complex. In particular, such assays are widely used in multiplexed formats to simultaneously analyze DNA samples at multiple loci, e.g. allele-specific multiplex PCR, arrayed primer extension (APEX) technology, solution phase primer extension or ligation assays, and the like, described in the following exemplary references: Syvanen, Nature Genetics Supplement, 37: S5-S10 (2005); Shumaker et al., Hum. Mut., 7: 346-354 (1996); Huang et al., U.S. Pat. Nos. 6,709,816 and 6,287,778; Fan et al., U.S. patent publication 2003/0003490; Gunderson et al., U.S. patent publication 2005/0037393; Hardenbol et al., Nature Biotechnology, 21: 673-678 (2003); Nilsson et al., Science, 265: 2085-2088 (1994); Baner et al., Nucleic Acids Research, 26: 5073-5078 (1998); Lizardi et al., Nat. Genet., 19: 225-232 (1998); Gerry et al., J. Mol. Biol., 292: 251-262 (1999); Fan et al., Genome Research, 10: 853-860 (2000); International patent publications WO 2002/57491 and WO 2000/58516; U.S. Pat. Nos. 6,506,594 and 4,883,750; and the like.

In one aspect, hybridization-based assays include circularizing probes, such as padlock probes, rolling circle probes, molecular inversion probes, linear amplification molecules for multiplexed PCR, and the like, e.g. padlock probes being disclosed in U.S. Pat. Nos. 5,871,921; 6,235,472; 5,866,337; and Japanese patent JP. 4-262799; rolling circle probes being disclosed in Aono et al., JP-4-262799; Lizardi, U.S. Pat. Nos. 5,854,033; 6,183,960; 6,344,239; molecular inversion probes being disclosed in Hardenbol et al. (supra) and in Willis et al., U.S. Pat. No. 6,858,412; and linear amplification molecules being disclosed in Faham et al., U.S. patent publication 2003/0104459. Such probes are desirable because non-circularized probes can be digested with single stranded exonucleases thereby greatly reducing background noise due to spurious amplifications, and the like. In the case of molecular inversion probes (MIPs), padlock probes, and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probe, probe concatemers, and the like, with an exonuclease, such as exonuclease I.

In certain exemplary embodiments, padlock probes are provided to profile the methylation state of a nucleic acid sample. As used herein, the term “padlock probe” includes, but is not limited to, an oligonucleotide sequence (e.g., about 70-140 nucleotides in length) that includes two regions of homology to a target nucleic acid sequence (e.g., genomic DNA) located at the termini or ends of the probe, two PCR primer regions, and two cleavage sites (See Hardenbol, Nature Biotech., Vol. 21, No. 6., 6 Jun. 1993, Hardenbol et al., Genome Research, 2005; 15(2):269-75; Fakhrai et al. (2003) Nature Biotech. 21(6):673 and Wang et al. (2005) Nucl. Acids Res. 33:e183). A padlock probe can be circularized by ligation in the presence of a correct target sequence. A universal detection tag sequence can be used for array detection of amplified probe. Cleavage sites are used to release the circularized probe from genomic DNA and for post-amplification processing.

Methods of conducting multiplexed hybridization-based assays using microarrays, and like platforms, suitable for the present invention are well known in the art. Guidance for selecting conditions and materials for applying labeled sequences to solid phase supports, such as microarrays, may be found in the literature, e.g. Wetmur, Crit. Rev. Biochem. Mol. Biol., 26: 227-259 (1991); DeRisi et al., Science, 278: 680-686 (1997); Chee et al., Science, 274: 610-614 (1996); Duggan et al., Nature Genetics, 21: 10-14 (1999); Schena, Editor, Microarrays: A Practical Approach (IRL Press, Washington, 2000); Freeman et al., Biotechniques, 29: 1042-1055 (2000); and like references. Methods and apparatus for carrying out repeated and controlled hybridization reactions have been described in U.S. Pat. Nos. 5,871,928, 5,874,219, 6,045,996 and 6,386,749, 6,391,623. Hybridization conditions typically include salt concentrations of less than about 1 M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e., conditions under which a probe will stably hybridize to a perfectly complementary target sequence, but will not stably hybridize to sequences that have one or more mismatches. The stringency of hybridization conditions depends on several factors, such as probe sequence, probe length, temperature, salt concentration, concentration of organic solvents, such as formamide, and the like. How such factors are selected is usually a matter of design choice to one of ordinary skill in the art for any particular embodiment. Usually, stringent conditions are selected to be about 5° C. lower than the Tₘ for the specific sequence for particular ionic strength and pH. Exemplary hybridization conditions include salt concentration of at least 0.01 M to about 1 M Na ion concentration (or other salts) at a pH 7.0 to 8.3 and a temperature of at least 25° C. Additional exemplary hybridization conditions include the following: 5×SSPE (750 mM NaCl, 50 mM sodium phosphate, 5 mM EDTA, pH 7.4).

Exemplary hybridization procedures for applying labeled target sequence to a GENFLEX™ microarray (Affymetrix, Santa Clara, Calif.) is as follows: denatured labeled target sequence at 95-100° C. for 10 minutes and snap cool on ice for 2-5 minutes. The microarray is pre-hybridized with 6×SSPE-T (0.9 M NaCl 60 mM NaH₂, PO₄, 6 mM EDTA (pH 7.4), 0.005% Triton X-100) +0.5 mg/ml of BSA for a few minutes, then hybridized with 120 μL hybridization solution (as described below) at 42° C. for 2 hours on a rotisserie at 40 RPM. Hybridization Solution consists of 3M TMACL (tetramethylammonium chloride), 50 mM MES ((2-[N-Morpholino]ethanesulfonic acid) Sodium Salt) (pH 6.7), 0.01% of Triton X-100, 0.1 mg/ml of herring sperm DNA, optionally 50 pM of fluorescein-labeled control oligonucleotide, 0.5 mg/ml of BSA (Sigma) and labeled target sequences in a total reaction volume of about 120 μL. The microarray is rinsed twice with 1×SSPE-T for about 10 seconds at room temperature, then washed with 1×SSPE-T for 15-20 minutes at 40° C. on a rotisserie at 40 RPM. The microarray is then washed 10 times with 6×SSPE-T at 22° C. on a fluidic station (e.g. model FS400, Affymetrix, Santa Clara, Calif.). Further processing steps may be required depending on the nature of the label(s) employed, e.g. direct or indirect. Microarrays containing labeled target sequences may be scanned on a confocal scanner (such as available commercially from Affymetrix) with a resolution of 60-70 pixels per feature and filters and other settings as appropriate for the labels employed. GENECHIP® (Affymetrix) or similar software may be used to convert the image files into digitized files for further data analysis.

Embodiments of the present invention are directed to the use of hybridization-based assays with polony sequencing technology or synthetic genomic technology. Polony technology is described in U.S. Pat. Nos. 6,432,360, 6,485,944 and 6,511,803 and PCT/US05/06425. In general, the term “polony” refers to “polymerized colony.” Polony technology relates to the amplification of nucleic acids. In general, a pool of nucleic acids is provided, preferably in an array where the nucleic acids are immobilized to a support. The nucleic acids are randomly patterned on the support. The nucleic acids are then amplified in situ to produce colonies of polymerized nucleic acids. Polony amplification can also take place on beads where a nucleic acid is attached to a bead and then polymerized in situ.

Samples or specimens containing target polynucleotides, such as fragments of genomic DNA, may come from a wide variety of sources for use with the present invention, including, but not limited to, cell cultures, animal or plant tissues, patient biopsies, environmental samples, and the like. Samples are prepared for assays of the invention using conventional techniques, which typically depend on the source from which a sample or specimen is taken.

Prior to carrying out reactions on a sample, it will often be desirable to perform one or more sample preparation operations upon the sample. Typically, these sample preparation operations will include such manipulations as extraction of intracellular material, e.g., nucleic acids from whole cell samples, viruses and the like.

For those embodiments where whole cells, viruses or other tissue samples are being analyzed, it will typically be necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations, e.g., denaturation of contaminating (DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced.

Following extraction, it will often be desirable to separate the nucleic acids from other elements of the crude extract, e.g., denatured proteins, cell membrane particles, salts, and the like. Removal of particulate matter is generally accomplished by filtration, flocculation or the like. A variety of filter types may be readily incorporated into the device. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample, passing salts through dialysis membranes, and the like. Suitable solid supports for nucleic acid binding include, e.g., diatomaceous earth, silica (i.e., glass wool), or the like. Suitable gel exclusion media, also well known in the art, may also be readily incorporated into the devices of the present invention, and is commercially available from, e.g., Pharmacia and Sigma Chemical.

In some applications, such as measuring target polynucleotides in rare cells from a patient's blood, an enrichment step may be carried out prior to conducting an assay, such as by immunomagnetic isolation, fluorescent cell sorting or other such technique. Such isolation or enrichment may be carried out using a variety of techniques and materials known in the art, as disclosed in the following representative references: Terstappen et al., U.S. Pat. No. 6,365,362; Terstappen et al., U.S. Pat. No. 5,646,001; Rohr et al., U.S. Pat. No. 5,998,224; Kausch et al., U.S. Pat. No. 5,665,582; Kresse et al., U.S. Pat. No. 6,048,515; Kausch et al., U.S. Pat. No. 5,508,164; Miltenyi et al., U.S. Pat. No. 5,691,208; Molday, U.S. Pat. No. 4,452,773; Kronick, U.S. Pat. No. 4,375,407; Radbruch et al., Chapter 23, in Methods in Cell Biology, Vol. 42 (Academic Press, New York, 1994); Uhlen et al., Advances in Biomagnetic Separation (Eaton Publishing, Natick, 1994); Safarik et al., J. Chromatography B, 722: 33-53 (1999); Miltenyi et al., Cytometry, 11: 231-238 (1990); Nakamura et al., Biotechnol. Prog., 17: 1145-1155 (2001); Moreno et al., Urology, 58: 386-392 (2001); Racila et al., Proc. Natl. Acad. Sci., 95: 4589-4594 (1998); Zigeuner et al., J. Urology, 169: 701-705 (2003); Ghossein et al., Seminars in Surgical Oncology, 20: 304-311 (2001).

In one aspect, genomic DNA for analysis is obtained using standard commercially available DNA extraction kits, e.g., PUREGENE® DNA Isolation Kit (Gentra Systems, Minneapolis, Minn.). In another aspect, for assaying human genomic DNA with a multiplex hybridization-based assay containing from about 1000 to 50,000 probes, a DNA sample may be used having an amount within the range of from about 200 ng to about 1 microgram. When sample material is scarce, prior to assaying, sample DNA may be amplified by whole genome amplification, or like technique, to increase the total amount of DNA available for assaying. Several whole genome, or partial genome, amplification techniques are known in the art, such as the following: Telenius et al. (1992) Genomics 13:718; Cheung et al. (1996) Proc. Natl. Acad. Sci. U.S.A. 93:14676; Dean et al. (2001) Genome Research 11:1095; U.S. Pat. Nos. 6,124,120; 6,280,949; 6,617,137; and the like.

Terms and symbols of nucleic acid chemistry, biochemistry, genetics, and molecular biology used herein follow those of standard treatises and texts in the field, e.g., Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Addressable” or “addressed” in reference to tag complements means that the nucleotide sequence, or perhaps other physical or chemical characteristics, of a tag complement can be determined from its address, i.e., a one-to-one correspondence between the sequence or other property of the tag complement and a spatial location on, or characteristic of, the solid phase support to which it is attached. In certain aspects, an address of a tag complement is a spatial location, e.g., the planar coordinates of a particular region containing copies of the tag complement. In other embodiments, probes may be addressed in other ways, e.g., by microparticle size, shape, color, color ratio or fluorescent ratio, radio frequency of micro-transponder, or the like, e.g., Kettman et al. (1998) Cytometry 33:234; Xu et al. (2003) Nucl. Acids Res. 31:e43; Bruchez Jr. et al., U.S. Pat. No. 6,500,622; Mandecki, U.S. Pat. No. 6,376,187; Stuelpnagel et al., U.S. Pat. No. 6,396,995; Chee et al., U.S. Pat. No. 6,544,732; Chandler et al., PCT publication WO 97/14028; and the like. According to the present invention, such terms also may refer to a nucleotide sequence that specifically identifies DNA or RNA sequences as having been captured from a given patient or other subject.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reaction (PCR), linear polymerase reactions, nucleic acid sequence-based amplification (NASBA), rolling circle amplifications, and the like, disclosed in the following references: Mullis et al., U.S. Pat. Nos. 4,683,195; 4,965,188; 4,683,202; 4,800,159 (PCR); Gelfand et al., U.S. Pat. No. 5,210,015 (real-time PCR with “Taqman” probes); Wittwer et al., U.S. Pat. No. 6,174,670; Kacian et al., U.S. Pat. No. 5,399,491 (“NASBA”); Lizardi, U.S. Pat. No. 5,854,033; Aono et al., Japanese Patent Pub. JP 4-262799 (rolling circle amplification); and the like. In one aspect, amplicons of the invention are produced by PCR. An amplification reaction may be a “real-time” amplification if a detection chemistry is available that permits a reaction product to be measured as the amplification reaction progresses, e.g. “real-time PCR” described below, or “real-time NASBA” as described in Leone et al. (1998) Nucl. Acids Res. 26:2150, and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like. Methods of “polony amplification” are also described in U.S. Pat. No. 6,432,360, U.S. Pat. No. 6,511,803 and U.S. Pat. No. 6,485,944.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucl. Acids Res. 12:203. According to the present invention, useful MIP primer sequences hybridize to sequences that flank the nucleotide base or series of bases to be captured.

“Complex” means an assemblage or aggregate of molecules in direct or indirect contact with one another. In one aspect, “contact,” or more particularly, “direct contact,” in reference to a complex of molecules or in reference to specificity or specific binding, means two or more molecules are close enough so that attractive noncovalent interactions, such as van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its component molecules. As used herein, “complex” refers to a duplex or triplex of polynucleotides or a stable aggregate of two or more proteins. In regard to the latter, a complex is formed by an antibody specifically binding to its corresponding antigen.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g., conditions including temperature of about 5° C. less that the Tₘ of a strand of the duplex and low monovalent salt concentration, e.g., less than 0.2 M, or less than 0.1 M. “Perfectly matched” in reference to a duplex means that the polynucleotide or oligonucleotide strands making up the duplex form a double stranded structure with one another such that every nucleotide in each strand undergoes Watson-Crick base pairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions. In another aspect, a genetic locus refers to the expressed nucleic acid product of a gene, such as an RNA molecule or a cDNA copy thereof.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Hybridization-based assay” means any assay that relies on the formation of a stable complex as the result of a specific binding event. In one aspect, a hybridization-based assay means any assay that relies on the formation of a stable duplex or triplex between a probe and a target nucleotide sequence for detecting or measuring such a sequence. In one aspect, probes of such assays anneal to (or form duplexes with) regions of target sequences in the range of from 8 to 100 nucleotides; or in other aspects, they anneal to target sequences in the range of from 8 to 40 nucleotides, or more usually, in the range of from 8 to 20 nucleotides. A “probe” in reference to a hybridization-based assay means a polynucleotide that has a sequence that is capable of forming a stable hybrid (or triplex) with its complement in a target nucleic acid and that is capable of being detected, either directly or indirectly. Hybridization-based assays include, without limitation, assays that use the specific base-pairing of one or more oligonucleotides as target recognition components, such as polymerase chain reactions, NASBA reactions, oligonucleotide ligation reactions, single-base extension reactions, circularizable probe reactions, allele-specific oligonucleotide hybridizations, either in solution phase or bound to solid phase supports, such as microarrays or microbeads, and the like. An important subset of hybridization-based assays include such assays that have at least one enzymatic processing step after a hybridization step. Hybridization-based assays of this subset include, without limitation, polymerase chain reactions, NASBA reactions, oligonucleotide ligation reactions, cleavase reactions, e.g., in INVADER™ assays, single-base extension reactions, probe circularization reactions, and the like. There is extensive guidance in the literature on hybridization-based assays, e.g., Hames et al., editors, Nucleic Acid Hybridization a Practical Approach (IRL Press, Oxford, 1985); Tijssen, Hybridization with Nucleic Acid Probes, Parts I & II (Elsevier Publishing Company, 1993); Hardiman, Microarray Methods and Applications (DNA Press, 2003); Schena, editor, DNA Microarrays a Practical Approach (IRL Press, Oxford, 1999); and the like. In one aspect, hybridization-based assays are solution phase assays; that is, both probes and target sequences hybridize under conditions that are substantially free of surface effects or influences on reaction rate. A solution phase assay includes circumstances where either probes or target sequences are attached to microbeads such that the attached sequences have substantially the same environment (e.g., permitting reagent access, etc.) as free sequences. In another aspect, hybridization-based assays include immunoassays wherein antibodies employ nucleic acid reporters based on amplification. In such assays, antibody probes specifically bind to target molecules, such as proteins, in separate reactions, after which the products of such reactions (i.e., antibody-protein complexes) are combined and nucleic acid reporters are amplified. Preferably, such nucleic acid reporters include oligonucleotide tags that are converted enzymatically into labeled oligonucleotide tags for analysis on a microarray, as described below. The following exemplary references disclose antibody-nucleic acid conjugates for immunoassays: Baez et al., U.S. Pat. No. 6,511,809; Sano et al., U.S. Pat. No. 5,665,539; Eberwine et al., U.S. Pat. No. 5,922,553; Landegren et al., U.S. Pat. No. 6,558,928; Landegren et al., U.S. Patent Pub. 2002/0064779; and the like. In particular, the two latter patent publications by Landegren et al. disclose steps of forming amplifiable probes after a specific binding event.

“Kit” refers to any delivery system for delivering materials or reagents for carrying out a method of the invention. In the context of assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. In one aspect, kits of the invention comprise probes specific for polymorphic loci. In another aspect, kits comprise nucleic acid standards for validating the performance of probes specific for polymorphic loci. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references: Whitely et al., U.S. Pat. No. 4,883,750; Letsinger et al., U.S. Pat. No. 5,476,930; Fung et al., U.S. Pat. No. 5,593,826; Kool, U.S. Pat. No. 5,426,180; Landegren et al., U.S. Pat. No. 5,871,921; Xu and Kool (1999) Nucl. Acids Res. 27:875; Higgins et al., Meth. in Enzymol. (1979) 68:50; Engler et al. (1982) The Enzymes, 15:3 (1982); and Namsaraev, U.S. Patent Pub. 2004/0110213.

“Microarray” refers in one embodiment to a type of multiplex assay product that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate creates a substantially planar surface. Spatially defined sites may additionally be “addressable” in that its location and the identity of the immobilized probe at that location are known or determinable. Probes immobilized on microarrays include nucleic acids, such as oligonucleotide barcodes, that are generated in or from an assay reaction. Typically, the oligonucleotides or polynucleotides on microarrays are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology relating to nucleic acid probes is reviewed in the following exemplary references: Schena, Editor, Microarrays: A Practical Approach (IRL Press, Oxford, 2000); Southern, Current Opin. Chem. Biol., 2: 404-410 (1998); Nature Genetics Supplement, 21:1-60 (1999); and Fodor et al., U.S. Pat. Nos. 5,424,186; 5,445,934; and 5,744,305. A microarray may comprise arrays of microbeads, or other microparticles, alone or disposed on a planar surface or in wells or other physical configurations that can be sued to separate the beads. Such microarrays may be formed in a variety of ways, as disclosed in the following exemplary references: Brenner et al. (2000) Nat. Biotechnol. 18:630; Tulley et al., U.S. Pat. No. 6,133,043; Stuelpnagel et al., U.S. Pat. No. 6,396,995; Chee et al., U.S. Pat. No. 6,544,732; and the like. In one format, microarrays are formed by randomly disposing microbeads having attached oligonucleotides on a surface followed by determination of which microbead carries which oligonucleotide by a decoding procedure, e.g. as disclosed by Gunderson et al., U.S. Patent Pub. No. 2003/0096239.

“Microarrays” or “arrays” can also refer to a heterogeneous pool of nucleic acid molecules that is distributed over a support matrix. The nucleic acids can be covalently or noncovalently attached to the support. Preferably, the nucleic acid molecules are spaced at a distance from one another sufficient to permit the identification of discrete features of the array. Nucleic acids on the array may be non-overlapping or partially overlapping. Methods of transferring a nucleic acid pool to support media is described in U.S. Pat. No. 6,432,360. Bead based methods useful in the present invention are disclosed in PCT US05/04373.

“Amplifying” includes the production of copies of a nucleic acid molecule of the array or a nucleic acid molecule bound to a bead via repeated rounds of primed enzymatic synthesis. “In situ” amplification indicated that the amplification takes place with the template nucleic acid molecule positioned on a support or a bead, rather than in solution. In situ amplification methods are described in U.S. Pat. No. 6,432,360.

“Support” can refer to a matrix upon which nucleic acid molecules of a nucleic acid array are placed. The support can be solid or semi-solid or a gel. “Semi-solid” refers to a compressible matrix with both a solid and a liquid component, wherein the liquid occupies pores, spaces or other interstices between the solid matrix elements. Semi-solid supports can be selected from polyacrylamide, cellulose, polyamide (nylon) and crossed linked agarose, dextran and polyethylene glycol.

“Randomly-patterned” or “random” refers to non-ordered, non-Cartesian distribution (in other words, not arranged at pre-determined points along the x- or y-axes of a grid or at defined “clock positions,” degrees or radii from the center of a radial pattern) of nucleic acid molecules over a support, that is not achieved through an intentional design (or program by which such design may be achieved) or by placement of individual nucleic acid features. Such a “randomly-patterned” or “random” array of nucleic acids may be achieved by dropping, spraying, plating or spreading a solution, emulsion, aerosol, vapor or dry preparation comprising a pool of nucleic acid molecules onto a support and allowing the nucleic acid molecules to settle onto the support without intervention in any manner to direct them to specific sites thereon. Arrays of the invention can be randomly patterned or random.

“Heterogeneous” refers to a population or collection of nucleic acid molecules that comprises a plurality of different sequences. According to one aspect, a heterogeneous pool of nucleic acid molecules results from a preparation of RNA or DNA from a cell which may be unfractionated or partially-fractionated.

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al., Exp. Opin. Ther. Patents, 6: 855-870 (1996); Mesmaeker et al., Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides comprising probes or primers usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

Usually nucleosidic monomers are linked by phosphodiester bonds. Whenever an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al., Molecular Cloning, Second Edition (Cold Spring Harbor Laboratory, New York, 1989), and like references. Oligonucleotides and polynucleotides may be single stranded or double stranded.

“Oligonucleotide tag” or “tag” means an oligonucleotide that is attached to a polynucleotide and is used to identify and/or track the polynucleotide in a reaction. Usually, an oligonucleotide tag is attached to the 3′- or 5′-end of a polynucleotide to form a linear conjugate, sometime referred to herein as a “tagged polynucleotide,” or equivalently, an “oligonucleotide tag-polynucleotide conjugate,” or “tag-polynucleotide conjugate.” Oligonucleotide tags may vary widely in size and compositions; the following references provide guidance for selecting sets of oligonucleotide tags appropriate for particular embodiments: Brenner, U.S. Pat. No. 5,635,400; Brenner et al., Proc. Natl. Acad. Sci., 97: 1665; Shoemaker et al. (1996) Nature Genetics, 14:450; Morris et al., EP Patent Pub. 0799897A1; Wallace, U.S. Pat. No. 5,981,179; and the like. I n different applications of the invention, oligonucleotide tags can each have a length within a range of from 4 to 36 nucleotides, or from 6 to 30 nucleotides, or from 8 to 20 nucleotides, respectively. A tag that is useful in the present invention to identify samples captured from a specific patient or other source is of sufficient length and complexity to distinguish it from sequences that identify other patients or sources of DNA being assayed in parallel. In one aspect, oligonucleotide tags are used in sets, or repertoires, wherein each oligonucleotide tag of the set has a unique nucleotide sequence. In some embodiment, particularly where oligonucleotide tags are used to sort polynucleotides, or where they are identified by specific hybridization, each oligonucleotide tag of such a set has a melting temperature that is substantially the same as that of every other member of the same set. In such aspects, the melting temperatures of oligonucleotide tags within a set are within 10° C. of one another; in another embodiment, they are within 5° C. of one another; and in another embodiment, they are within 2° C. of one another. In another aspect, oligonucleotide tags are members of a minimally cross-hybridizing set. That is, the nucleotide sequence of each member of such a set is sufficiently different from that of every other member of the set that no member can form a stable duplex with the complement of any other member under stringent hybridization conditions. In one aspect, the nucleotide sequence of each member of a minimally cross-hybridizing set differs from those of every other member by at least two nucleotides. Such a set of oligonucleotide tags may have a size in the range of from two, three, four, five etc., up to ten and several tens to many thousands, or even millions, e.g., 50 to 1.6×10⁶. In another embodiment, such a size is in the range of from 200 to 40,000; or from 200 to 40,000; or from 200 to 10,000.

In one embodiment, an amplifiable probe of the invention comprises at least one oligonucleotide tag that is replicated and labeled to produce a labeled oligonucleotide probe. Alternatively, where patient specific tags are envisioned the tag can be detected by stringent hybridization or alternatively sequenced along with the target sequence. In one embodiment, labeled oligonucleotide probes are hybridized to a microarray of tag complements for detection. In this embodiment, for each different locus of each different genome (e.g., from distinct patients, patient samples or other sources) there is a unique labeled oligonucleotide tag. That is, the pair consisting of (i) the nucleotide sequence of the oligonucleotide tag and (ii) a label that generates detectable signal are uniquely associated with a particular locus of a particular genome. The nature of the label on an oligonucleotide tag can be based on a wide variety of physical or chemical properties including, but not limited to, light absorption, fluorescence, chemiluminescence, electrochemiluminescence, mass, charge, and the like. The signals based on such properties can be generated directly or indirectly. For example, a label can be a fluorescent molecule covalently attached to an amplified oligonucleotide tag that directly generates an optical signal. Alternatively, a label can comprise multiple components, such as a hapten-antibody complex, that, in turn, may include fluorescent dyes that generated optical signals, enzymes that generate products that produce optical signals, or the like. In certain aspects, the label on an oligonucleotide tag is a fluorescent label that is directly or indirectly attached to an amplified oligonucleotide tag. In one aspect, such fluorescent label is a fluorescent dye or quantum dot selected from a group consisting of from 2 to 6 spectrally resolvable fluorescent dyes or quantum dots. In a different embodiment, a set of samples could be queried serially, i.e. using one tag at a time, with each of the tags that represent different patients, samples, etc., wherein each tag is labeled with the same label, and what is detected is binding or no binding to members of the set of samples, thereby identifying in each round a given patient's sample.

Fluorescent labels and their attachment to oligonucleotides, such as oligonucleotide tags, are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26:227-259 (1991); and the like. Particular methodologies applicable to the invention are disclosed in the following sample of references: Fung et al., U.S. Pat. No. 4,757,141; Hobbs, Jr., et al. U.S. Pat. No. 5,151,507; Cruickshank, U.S. Pat. No. 5,091,519. In one aspect, one or more fluorescent dyes are used as labels for labeled target sequences, e.g., as disclosed by Menchen et al., U.S. Pat. No. 5,188,934 (4,7-dichlorofluorscein dyes); Begot et al., U.S. Pat. No. 5,366,860 (spectrally resolvable rhodamine dyes); Lee et al., U.S. Pat. No. 5,847,162 (4,7-dichlororhodamine dyes); Khanna et al., U.S. Pat. No. 4,318,846 (ether-substituted fluorescein dyes); Lee et al., U.S. Pat. No. 5,800,996 (energy transfer dyes); Lee et al., U.S. Pat. No. 5,066,580 (xanthine dyes): Mathies et al., U.S. Pat. No. 5,688,648 (energy transfer dyes); and the like. Labelling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like. As used herein, the term “fluorescent label” includes a signaling moiety that conveys information through the fluorescent absorption and/or emission properties of one or more molecules. Such fluorescent properties include fluorescence intensity, fluorescence life time, emission spectrum characteristics, energy transfer, and the like.

Commercially available fluorescent nucleotide analogues readily incorporated into the labeling oligonucleotides include, for example, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY TM 630/650-14-dUTP, BODIPY TM 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY TM FL-14-UTP, BODIPY TMR-14-UTP, BODIPY TM TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.). Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000).

Other fluorophores available for post-synthetic attachment include, inter alia, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), and Cy2, Cy3.5, Cy5.5, and Cy7 (Amersham Biosciences, Piscataway, N.J. USA, and others).

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver particles may be coated onto the surface of the array to enhance signal from fluorescently labeled oligos bound to the array. Lakowicz et al. (2003) BioTechniques 34:62.

Biotin, or a derivative thereof, may also be used as a label on a detection oligonucleotide, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into a detection oligonucleotide and subsequently coupled to an N-hydroxy succinimide (NHS) derivatized fluorescent dye, such as those listed supra. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for detection oligonucleotides may include fluorescein (FAM), digoxigenin, dinitrophenol (DNP), dansyl, biotin, bromodeoxyuridine (BrdU), hexahistidine (6× His), phosphor-amino acids (e.g. P-tyr, P-ser, P-thr), or any other suitable label. In one embodiment the following hapten/antibody pairs are used for detection, in which each of the antibodies is derivatized with a detectable label: biotin/α-biotin, digoxigenin/a-digoxigenin, dinitrophenol (DNP)/α-DNP, 5-Carboxyfluorescein (FAM)/α-FAM.

As mentioned above, oligonucleotide tags can be indirectly labeled, especially with a hapten that is then bound by a capture agent, e.g., as disclosed in Holtke et al., U.S. Pat. Nos. 5,344,757; 5,702,888; and 5,354,657; Huber et al., U.S. Pat. No. 5,198,537; Miyoshi, U.S. Pat. No. 4,849,336; Misiura and Gait, PCT publication WO 91/17160; and the like. Many different hapten-capture agent pairs are available for use with the invention, either with a target sequence or with a detection oligonucleotide used with a target sequence, as described below. Exemplary, haptens include, biotin, des-biotin and other derivatives, dinitrophenol, dansyl, fluorescein, CY5, and other dyes, digoxigenin, and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C.

The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g., 200 nL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al. (1999) Anal. Biochem., 273:221-228 (two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); Becker-Andre et al., Nucleic Acids Research, 17:9437-9446 (1989); and the like.

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual). Usually, insertions or deletions are with respect to a major allele at a locus within a population, e.g., an allele present in a population at a frequency of fifty percent or greater.

“Primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention can be universal primers or non-universal primers. Pairs of primers can flank a sequence of interest or a set of sequences of interest. Primers and probes can be degenerate in sequence. Primers within the scope of the present invention bind adjacent to the target sequence, whether it is the sequence to be captured for analysis, or a tag that it to be copied.

“Solid support,” “support,” and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide. Semisolid supports and gel supports are also useful in the present invention, especially when polony amplification is used.

“Specific” or “specificity” in reference to the binding of one molecule to another molecule, such as a target sequence to a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as van der Waal forces, hydrogen bonding, base-stacking interactions, ionic and hydrophobic interactions, and the like, dominate the interaction of the molecules.

“Spectrally resolvable” in reference to a plurality of fluorescent labels means that the fluorescent emission bands of the labels are sufficiently distinct, i.e., sufficiently non-overlapping, that molecular tags to which the respective labels are attached can be distinguished on the basis of the fluorescent signal generated by the respective labels by standard photodetection systems, e.g., employing a system of band pass filters and photomultiplier tubes, or the like, as exemplified by the systems described in U.S. Pat. Nos. 4,230,558; 4,811,218, or the like, or in Wheeless et al., pgs. 21-76, in Flow Cytometry: Instrumentation and Data Analysis (Academic Press, New York, 1985). In one aspect, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

“Tₘ” is used in reference to “melting temperature.” Melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation. Tₘ=81.5+0.41 (% G+C), when a nucleic acid is in aqueous solution at 1 M NaCl (see e.g., Anderson and Young, “Quantitative Filter Hybridization,” in Nucleic Acid Hybridization (1985). Other references (e.g., Allawi, H. T. & Santa Lucia, J., Jr., Biochemistry 36, 10581-94 (1997)) include alternative methods of computation which take structural and environmental, as well as sequence characteristics into account for the calculation of Tₘ.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool or tissue), as well as liquid and solid food and feed products and ingredients such as dairy items, vegetables, meat and meat by-products, and waste. Biological samples may include materials taken from a patient including, but not limited to cultures, cells, tissues, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, tables, and accompanying claims.

### Example I

### Bisulfite Padlock Probes (BSPPs)

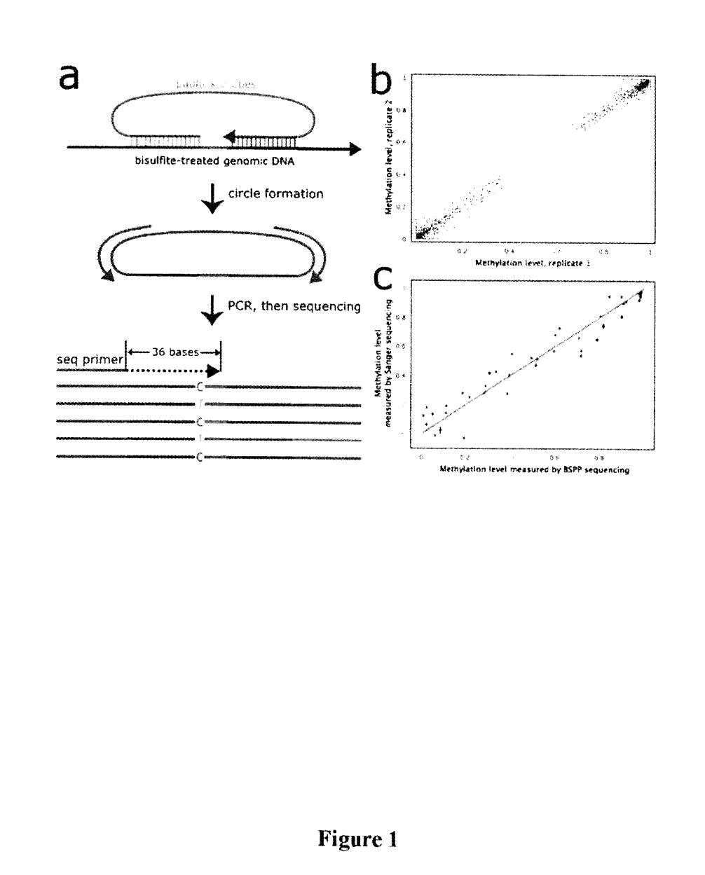

Bisulfite padlock probe (BSPP) technology, is a targeted method that isolates selected locations for methylation profiling. In this example, a “padlock probe” refers a probe that was an approximately 100 nucleotide DNA fragment that was designed to hybridize to genomic DNA targets in a horseshoe manner (FIG. 1A) (Nilsson, M. et al., Padlock probes: circularizing oligonucleotides for localized DNA detection. Science 265 (5181), 2085-2088 (1994); Hardenbol, P. et al., Multiplexed genotyping with sequence-tagged molecular inversion probes. Nat Biotechnol 21 (6), 673-678 (2003); Porreca et al., Multiplex amplification of large sets of human exons. Nat Methods 4 (11), 931-936 (2007)). The gap between the two hybridized, locus-specific arms of a padlock probe is polymerized and ligated to form a circular strand of DNA. These circles can then be amplified using the common “backbone” sequence that connects the two arms. This makes padlock probes highly multiplexable, with tens of thousands of probes used within a single reaction. The resulting libraries can then be sequenced with a massively parallel sequencing system. Padlock probes have been successfully used to specifically amplify 10,000 human exons (Porreca et al., supra), and an over 10,000-fold improvement in capturing efficiency has been made.

To apply padlock probes to profiling DNA methylation, a probe set was designed to target 10,000 locations in a bisulfite-treated human genome (Example IV and Table 1). Bisulfite treatment converts all unmethylated cytosines to uracil, which is recognized as a thymine (Clark, S. J., Harrison, J., Paul, C. L., & Frommer, M., High sensitivity mapping of methylated cytosines. Nucleic Acids Res 22 (15), 2990-2997 (1994)). These probes targeted the ENCODE regions, which represent ˜1% of the human genome and for which expression and chromatin immunoprecipitation (ChIP) data are available (Birney, E. et al., Identification and analysis of functional elements in 1% of the human genome by the ENCODE pilot project. Nature 447 (7146), 799-816 (2007)). Rather than targeting promoter regions or CpG islands, these probes were distributed randomly over all ENCODE regions (Table 2).

TABLE 1
BSPP list (separate file) and description of columns. Table 1 contains the
sequences of the bisulfite padlock probes designed for one experiment,
along with additional information and experimental data. “Estimated
methylation” values only existed if there were 10 or more observations
of a site, otherwise “NA” was given.

| Column | Description |
|---|---|
| 1 | ENCODE target region ID |
| 2 | bisulfite padlock probe sequence |
| 3 | chromosome target |
| 4 | start position of 10 bp targeted span |
| 5 | end position of 10 bp targeted span |
| 6 | strand |
| 7 | position(s) of targeted CpG cytosines (comma separated |
| | if more than one) |
| 8 | GM06990 technical replicate 1: number of observations |
| 9 | GM06990 technical replicate 1: estimated methylation |
| 10 | GM06990 technical replicate 2: number of observations |
| 11 | GM06990 technical replicate 2: estimated methylation |
| 12 | PGP1 lymphocyte: number of observations |
| 13 | PGP1 lymphocyte: estimated methylation |
| 14 | PGP9 lymphocyte: number of observations |
| 15 | PGP9 lymphocyte: estimated methylation |
| 16 | PGP1 fibroblast: number of observations |
| 17 | PGP1 fibroblast: estimated methylation |
| 18 | PGP9 fibroblast: number of observations |
| 19 | PGP9 fibroblast: estimated methylation |
| 20 | PGP1 induced pluripotent cells: number of observations |
| 21 | PGP1 induced pluripotent cells: estimated methylation |
| 22 | PGP9 induced pluripotent cells clone 1: number of observations |
| 23 | PGP9 induced pluripotent cells clone 1: estimated methylation |
| 24 | PGP9 induced pluripotent cells clone 2: number of observations |
| 25 | PGP9 induced pluripotent cells clone 2: estimated methylation |

TABLE 2
Distribution statistics for BSPP probes and MSCC sites. This table
gives some statistics on the sites profiled by BSPP and MSCC
methods, and compares these to hypothetical profiles of “all CpG
sites” and “all genomic sequence.”

| | BSPP | MSCC | | genomic |
|---|---|---|---|---|
| | (ENCODE set) | (unique HpaII) | all CpG sites | sequence |
| number | 9,552 probes | 1,417,432 | 28,485,346 | NA |
| | (10,704 CpGs) | | | |
| within CpG | 1.2% | 13.5% | 7.5% | 0.7% |
| islands | | | | |
| within 1 kb | 5.7% | 3.4% | 2.3% | 1.3% |
| of TSS | | | | |
| inside genes | 54.6% | 47.8% | 43.3% | 34.3% |
| within | 0% (by design) | 33.5% | 51.5% | 48.8% |
| repetitive | | | | |
| sequence | | | | |

All data was produced using the March 2006 human reference sequence (NCBI Build 36.1), downloaded from University of California at Santa Cruz (UCSC). CpG islands were based on UCSC's CpG island annotation. Transcription start sites (TSS) and gene locations were calculated using UCSC's RefGene list. Repetitive sequence was based on the letter casing in the genome sequence, produced by UCSC.

Without intending to be bound by scientific theory, given that no effort was made to target gene regions in the design, it seemed unlikely that 54.6% of BSPP probes were within genes. However, this is consistent with the fact that approximately 60% of ENCODE regions are in gene transcript regions by calculations that were performed (based on RefGene annotations). To simplify design, the BSPP probes avoided targeting sites with CpGs in the hybridizing arms: approximately 60% of all CpG sites and approximately 98% of CpGs within CpG islands were excluded by this criterion alone from potentially being assayed.

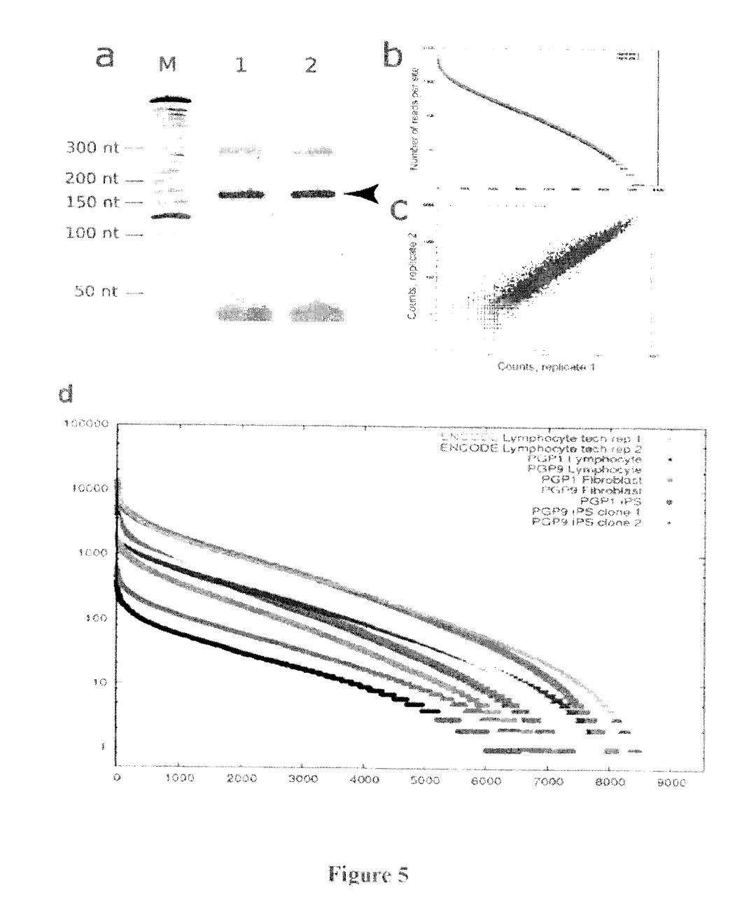

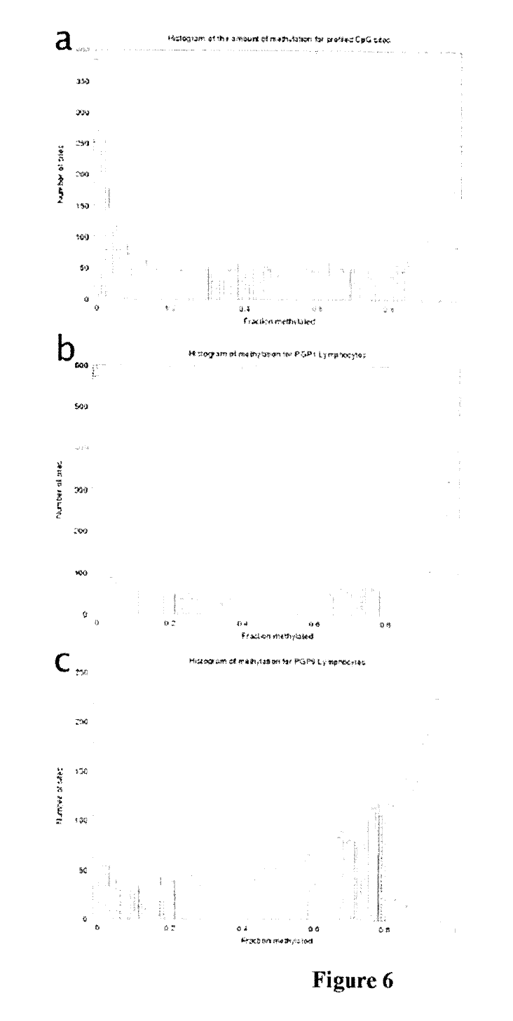

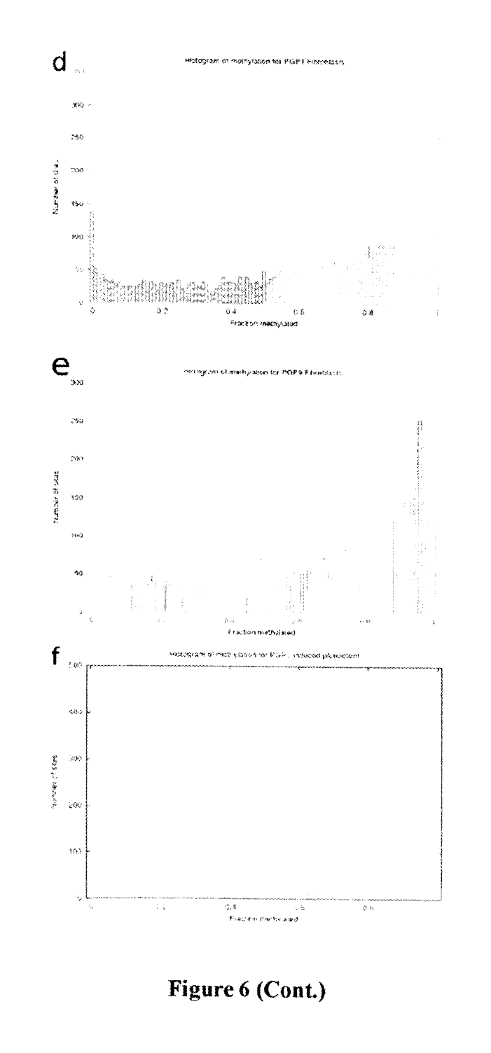

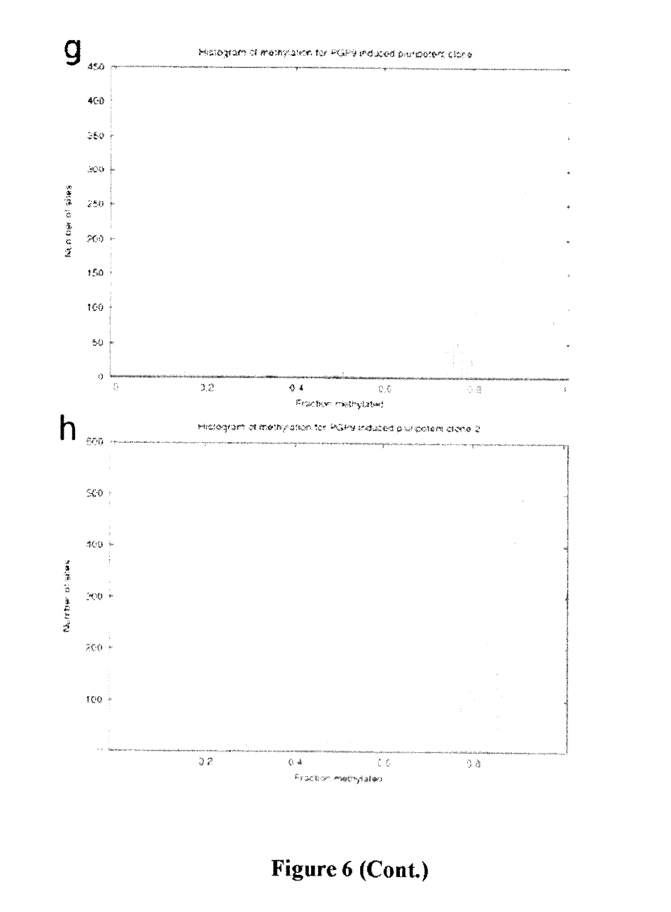

An initial experiment used the BSPP set to investigate cytosine methylation in the GM06990 EBV-transformed B-lymphocyte cell line, a cell line also used in the ENCODE project (Birney, E. et al., supra) (Example IV). The expected size band was observed and isolated from the gel (FIG. 5A) and, to check the specificity of the capturing, 75 individual library molecules were cloned and sequenced. All were unique and mapped to the desired target regions, illustrating the high specificity padlock probe technology could achieve despite the reduced genomic complexity after bisulfite conversion. Technical replicates of capturing were performed followed by Illumina Genome Analyzer (formerly Solexa) sequencing to check the reproducibility of the method and found that both the numbers of probe observations (FIGS. 5B and 5C) and the inferred methylation levels (FIG. 1B) were highly correlated. To rule out the possibility of systemic bias, traditional Sanger sequencing was performed on 33 regions amplified from bisulfite treated DNA. The methylation levels determined by this method were highly correlated with the BSPP-determined methylation (FIG. 1C and Example IV). Methylation levels were bimodally distributed with most sites <20% or >80% methylated (FIG. 6A), which is consistent with previous reports (Eckhardt, F. et al., DNA methylation profiling of human chromosomes 6, 20 and 22. Nat Genet 38 (12), 1378-1385 (2006)).

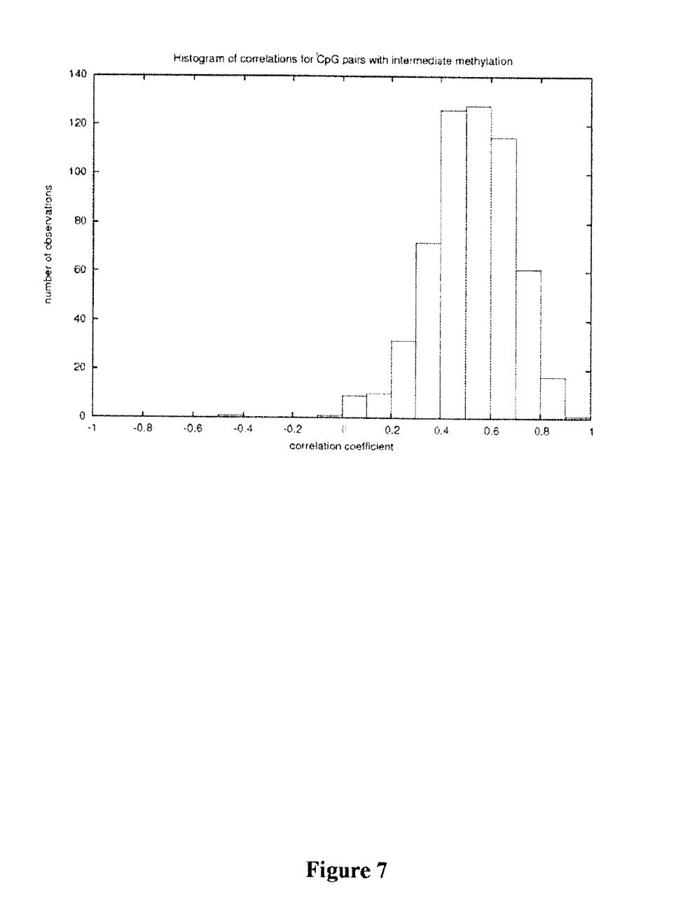

Because the sequencing was clonal, BSPP data could be used to investigate strand-specific information (e.g., correlation with neighboring CpG sites or SNPs). It was determined that, within probes spanning more than one CpG, there was a positive correlation between the methylation states of those CpGs on individual strands (FIG. 7).

### Example II

### Methyl Sensitive Cut Counting (MSCC)

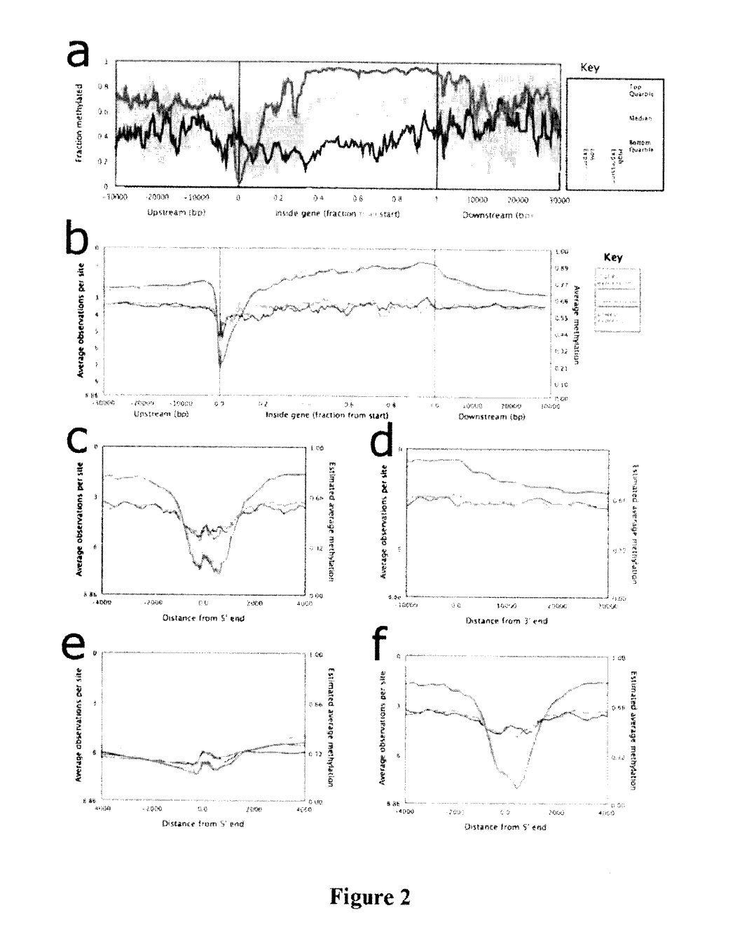

To explore the relationship between methylation and gene expression levels in the promoter region and elsewhere in the gene, ENCODE project gene expression data was used for this cell line to split genes into two equal groups: “highly expressed” and “lowly expressed” genes. For each group plotted, median cytosine methylation was plotted against gene position (FIG. 2A). In the highly expressed genes, a pattern of low methylation was observed in the promoter region and high methylation was observed in the rest of the gene body. The lowly expressed genes had moderate methylation in both promoter and gene-body regions.

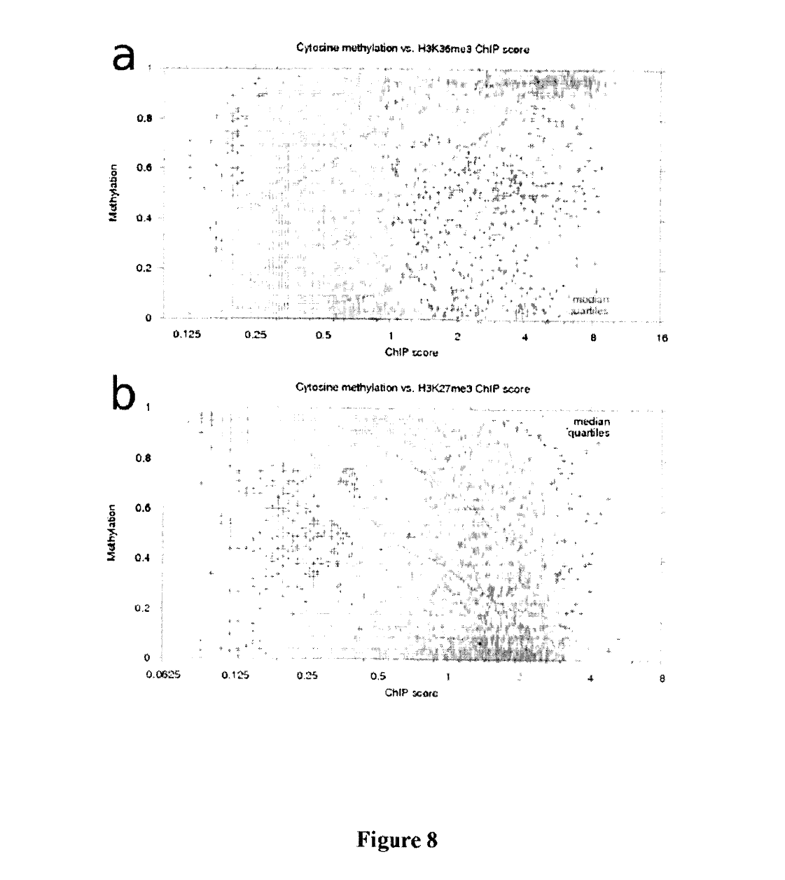

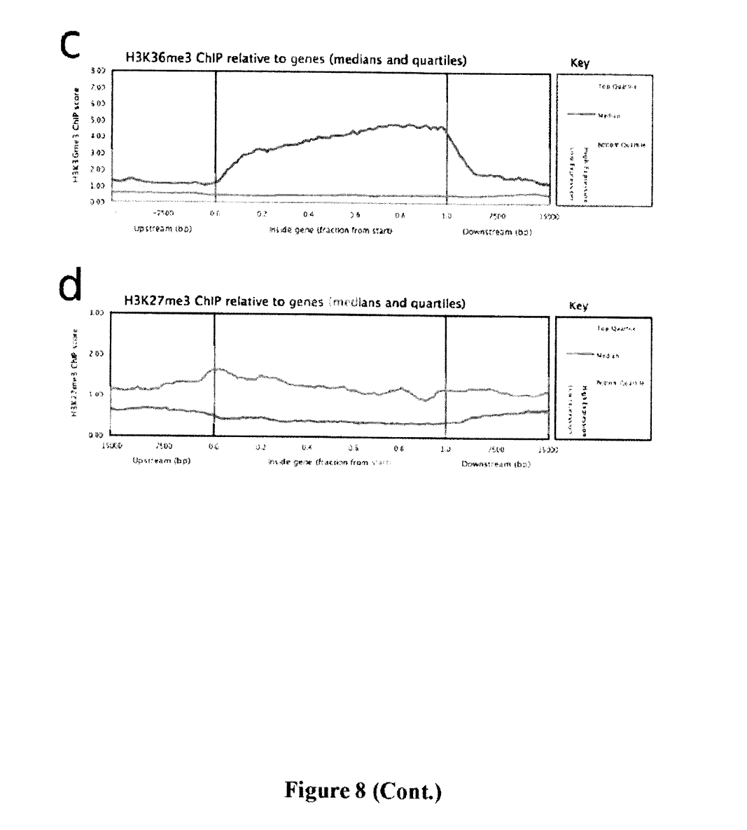

Without intending to be bound by scientific theory, cytosine methylation is an epigenetic feature that may interact with other epigenetic features such as histone modifications. To look for correlations between DNA methylation and histone modification, available ChIP data (Birney, E. et al., Identification and analysis of functional elements in 1% of the human genome by the ENCODE pilot project. Nature 447 (7146), 799-816 (2007)) was compared with the methylation presented herein. Cytosine methylation was observed and was correlated with H3K36 methylation and anti-correlated with H3K27 methylation (FIG. 8). Without intending to be bound by scientific theory, these correlations likely reflect the distribution of the probes, half of which fell within gene bodies (only 5% were within 1 kb of transcription start sites). The correlations were consistent with the gene-body pattern of the histone modifications: H3K36 methylation is higher in the gene-body of highly expressed genes, while H3K27 is high in the gene-body of lowly expressed genes (Barski, A. et al., High-resolution profiling of histone methylations in the human genome. Cell 129 (4), 823-837 (2007)).

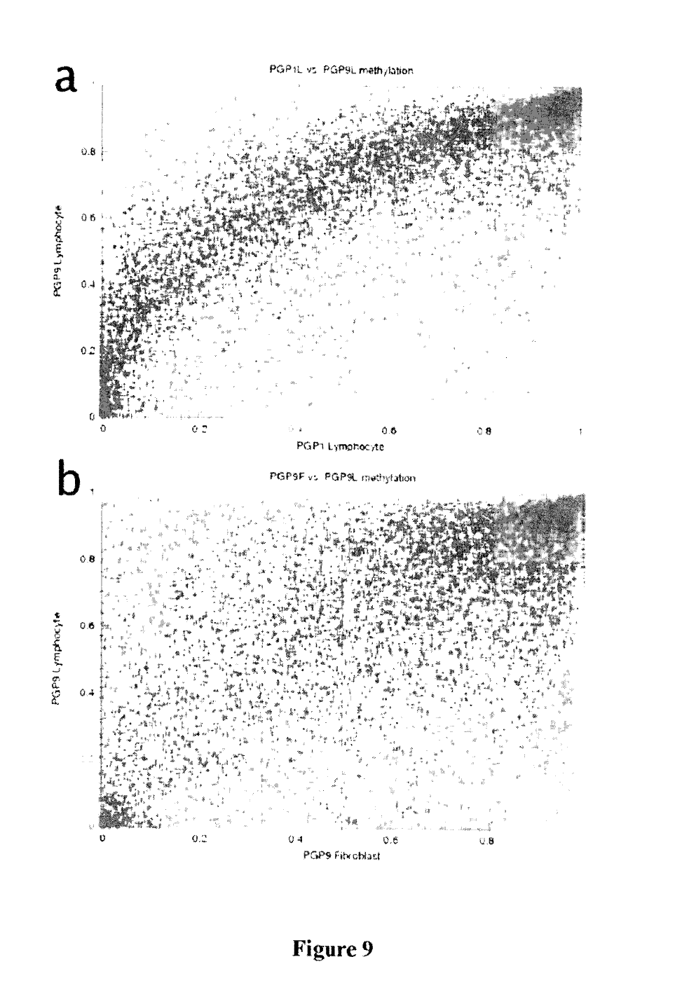

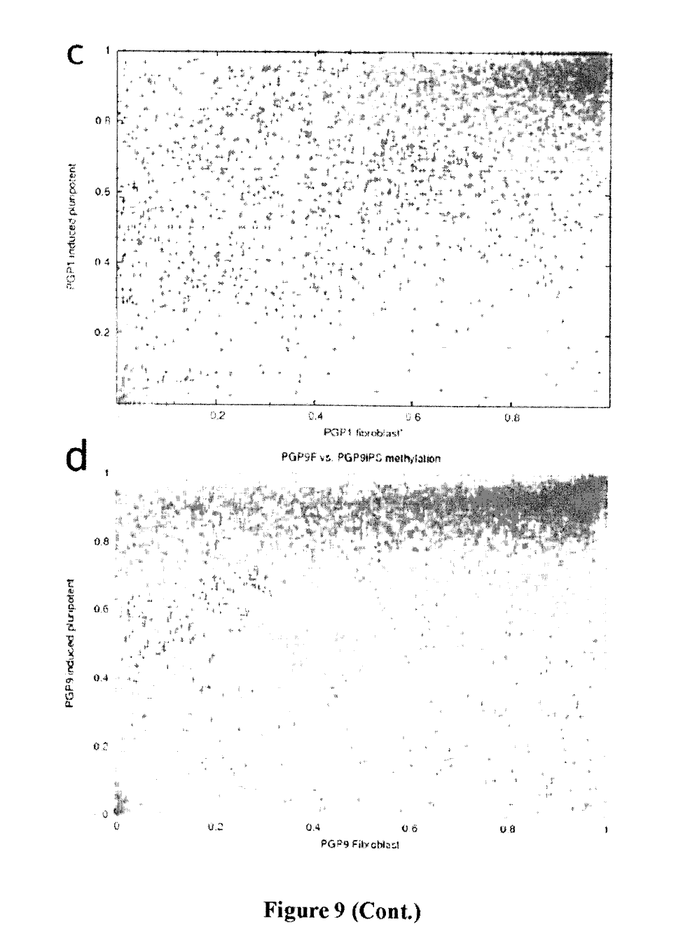

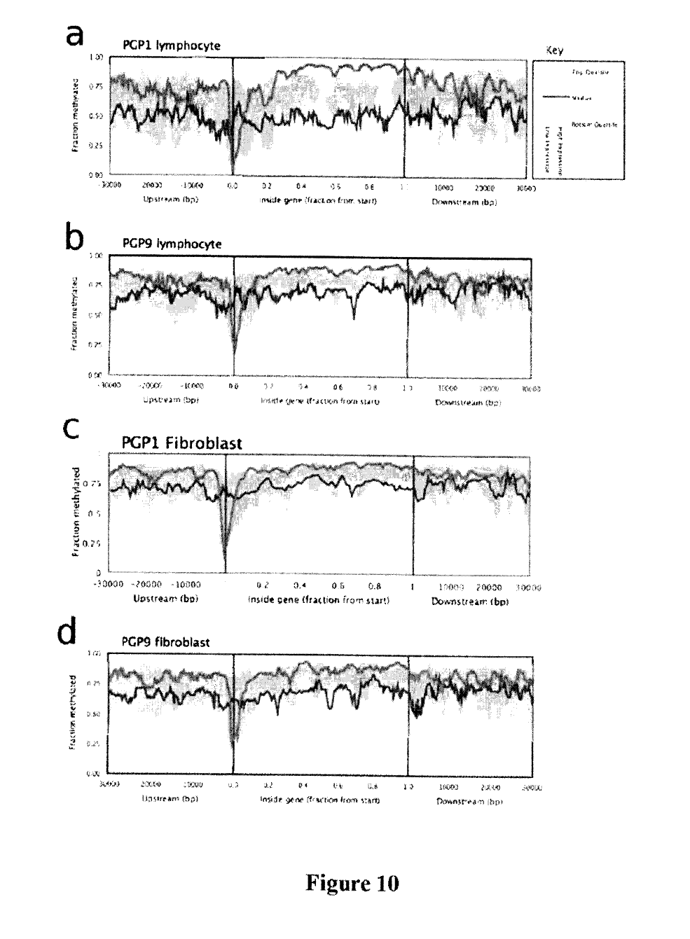

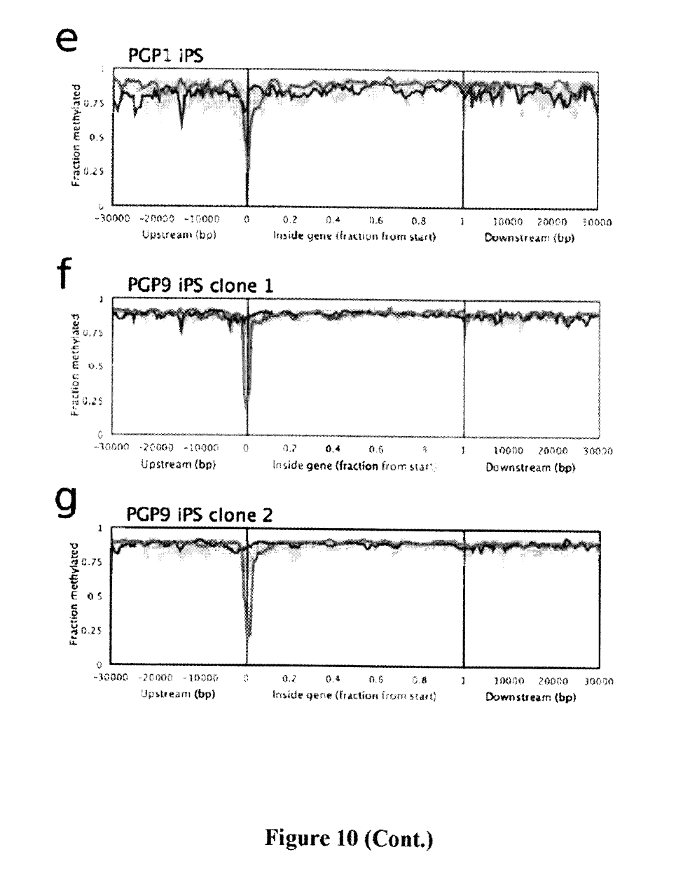

The methylation profiling methods will, in part, be used to deeply explore the relationship between genotype and phenotype through collection of multi-faceted biological information for individuals registered within the personal genome project (Church, G. M., The personal genome project. Mol Syst Biol 1, 2005 0030 (2005)). To explore how methylation patterns vary between different cell types and different individuals, the ENCODE BSPP set was applied to several cell lines from the PGP: PGP1 EBV-transformed B-lymphocytes, PGP9 EBV-transformed B-lymphocytes, PGP9 fibroblasts, and induced pluripotent stem cells (iPS) derived from PGP9 fibroblasts (Section IV). Consistent with previous studies (Eckhardt, F. et al., DNA methylation profiling of human chromosomes 6, 20 and 22. Nat Genet 38 (12), 1378-1385 (2006)), the methylation patterns of lymphoblast lines derived from different individuals were highly correlated (FIG. 9A), while the correlation between fibroblast and lymphoblast cells from the same individual was much lower (FIG. 9B). The PGP9 iPS cells were hypermethylated in the ENCODE regions of ˜400 genes, compared to the fibroblast line they were derived from (FIG. 9C). Further investigation is needed as cell culturing can affect global methylation levels (Meissner, A. et al., Genome-scale DNA methylation maps of pluripotent and differentiated cells. Nature 454 (7205), 766-770 (2008)). Using gene expression data described herein, it was observed that the phenomenon of gene-body methylation was repeated in the PGP cell lines (Example IV and FIG. 10).

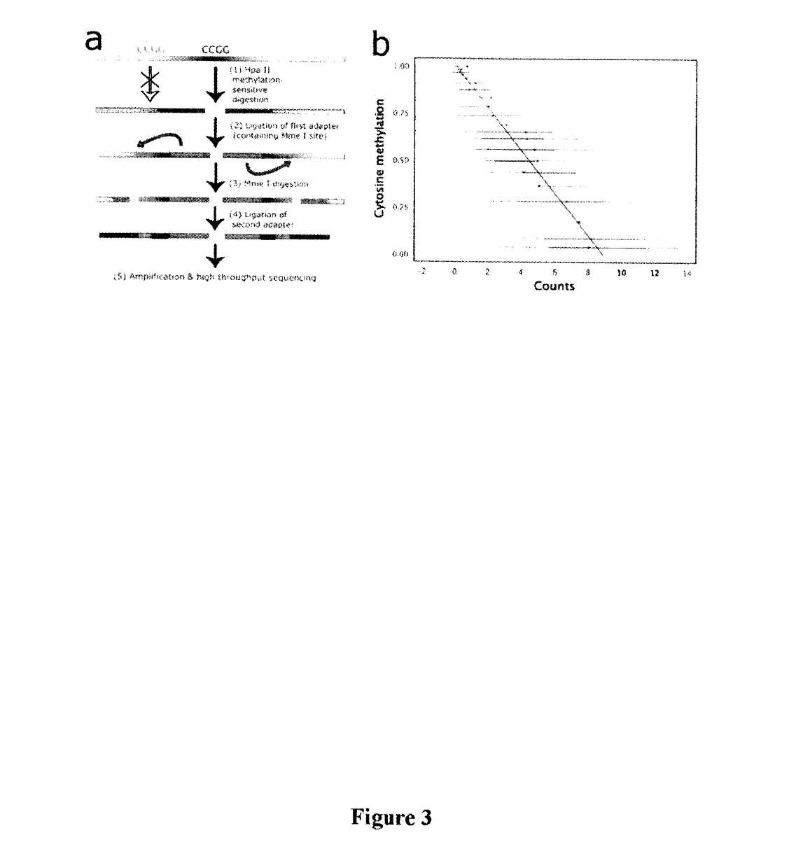

Methyl sensitive cut counting (MSCC), is a whole genome methylation profiling method. MSCC queries the sensitivity of all CCGG sites within the genome to HpaII, a methylation sensitive restriction enzyme that cuts at CCGG sequences. Methylation sensitive restriction enzymes are a common tool for studying methylation: these enzymes typically have a recognition site that contains a CpG dinucleotide and are blocked from cutting if that site is methylated (Bird, A. P. & Southern, E. M., Use of restriction enzymes to study eukaryotic DNA methylation: I. The methylation pattern in ribosomal DNA from Xenopus laevis. J Mol Biol 118 (1), 27-47 (1978)). With MSCC, no choice is made for which sites are targeted—all uniquely identifiable HpaII sites are profiled. By generating a library of tag fragments from all cut locations and then using massively parallel sequencing to gather millions of observations of these, one of skill in the art can infer the methylation level by the number of tags observed (FIG. 3A). Sites with many v. no reads were inferred to have low or high methylation levels, respectively. A control library was also constructed by replacing HpaII with a methylation-insensitive isoschizomer, MspI. However, the additional cost incurred does not seem warranted, as the data indicated that the HpaII library alone was highly correlated with methylation at individual sites (see below).

The human genome contains 2.3 million HpaII sites and each of these, if cut, can generate two possible library tags. Of the 4.6 million possible sequences, about half (2.3 million) are considered “unique,” i.e., they have more than one base difference when compared to any other possible sequence. Of the 2.3 million sites, 888,455 produce two unique tags and 528,977 produce a single unique tag. These combine to a total of 1,417,432 genomic locations that are profiled with this method. Nearly half of these sites occur within genes (>18,000 genes have at least one site within them) and 13.5% are within CpG islands (90% of CpG islands have at least one site within them) (Table 2).

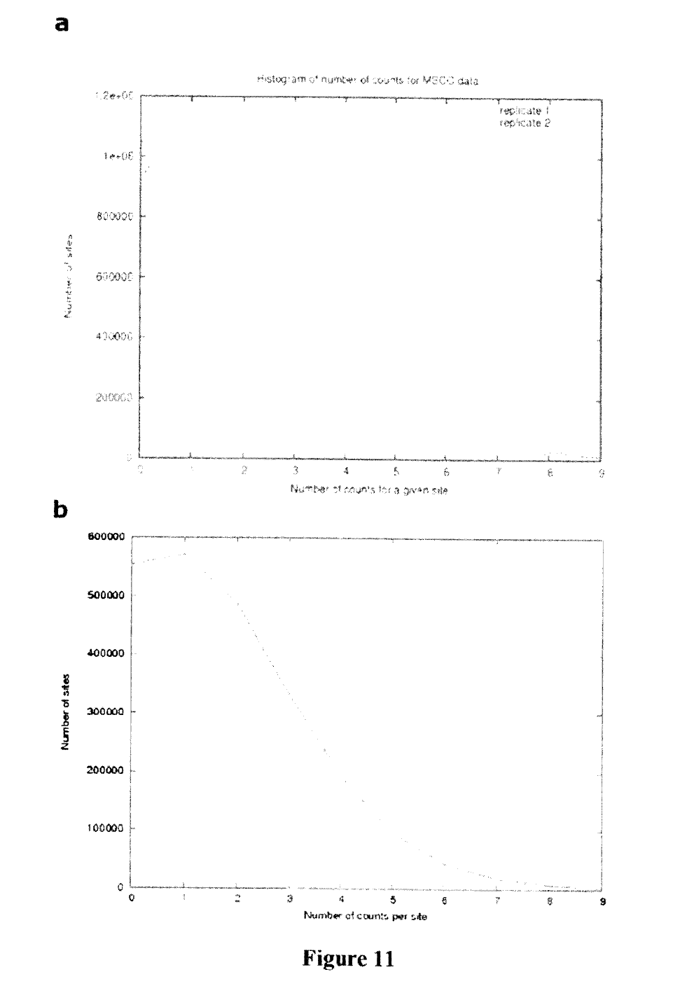

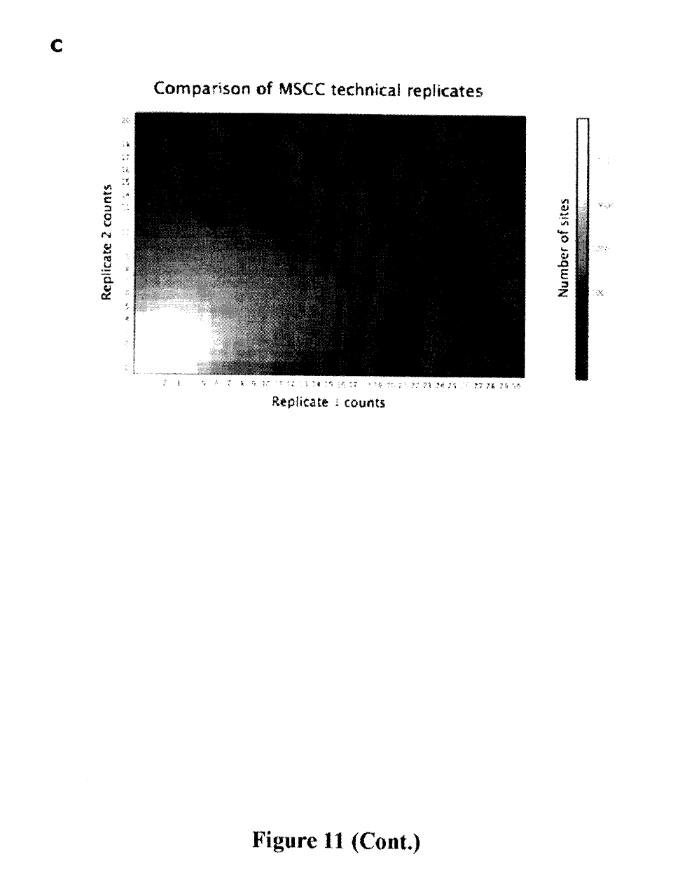

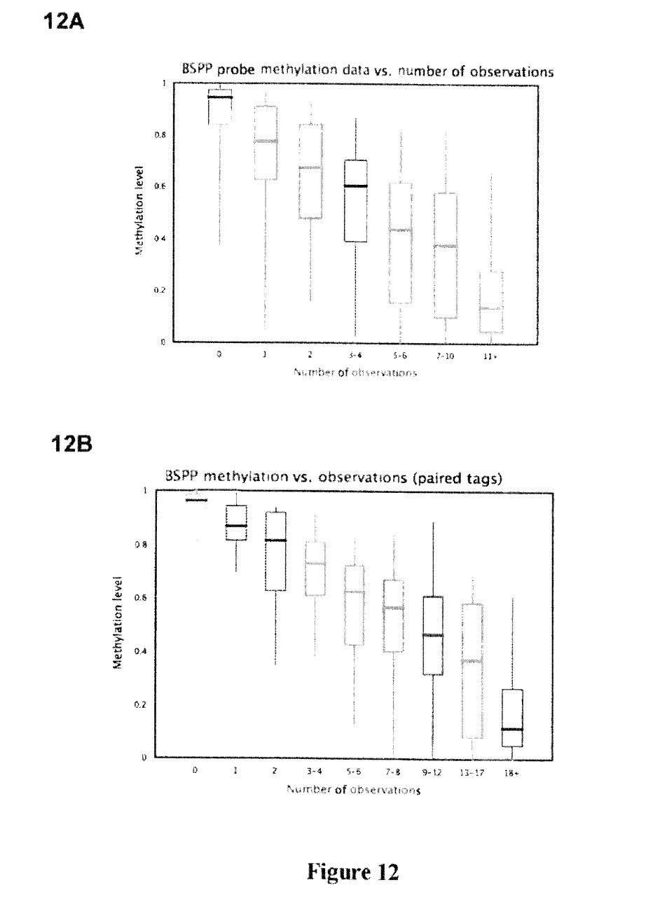

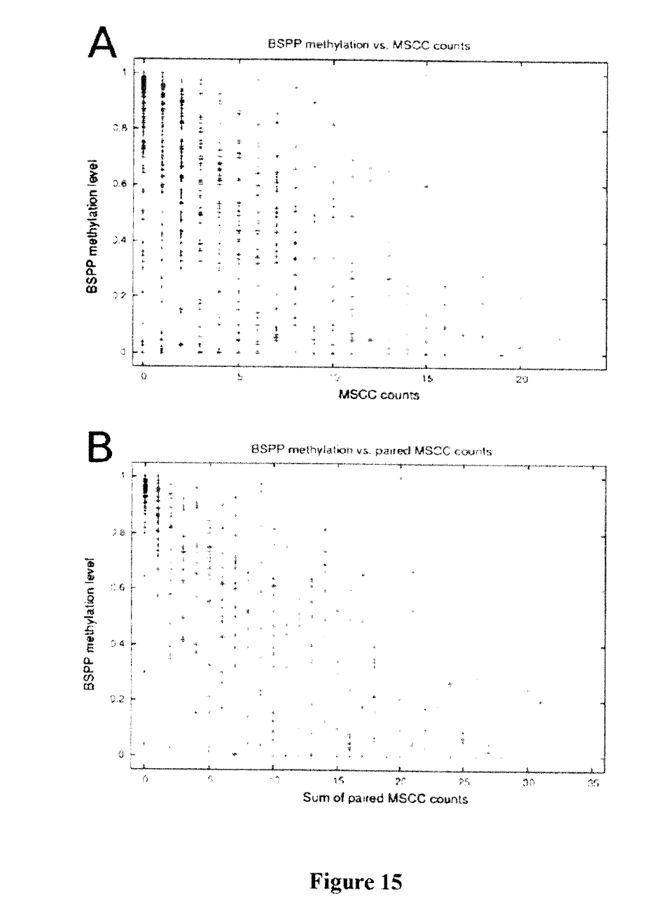

An MSCC HpaII library and an MspI control library were produced for the PGP1 EBV-transformed B-lymphocyte cell line, for which BSPP and gene expression data had been obtained. Libraries were sequenced using an Illumina Genome Analyzer and matched to a list of all possible tag sequences (Table 5). Two technical replicates were made of the HpaII library that, although subject to variance according to the Poisson distribution, showed a high correlation in the number of observations for each site (R=0.82, FIG. 11). The availability of BSPP data for the same sample enabled comparison of the methylation levels determined by BSPPs with MSCC HpaII data for 381 sites (726 individual tags) (FIG. 15). When data were binned according to the BSPP-determined methylation levels, the average number of counts for each bin was linearly related to its methylation level (FIG. 3B). This was used to estimate average methylation levels when counts for multiple sites are averaged. BSPP methylation data could also be used to estimate methylation levels for individual sites based on MSCC HpaII counts (FIG. 12).

MSCC counts had more noise for sites containing more than one HpaII recognition site.

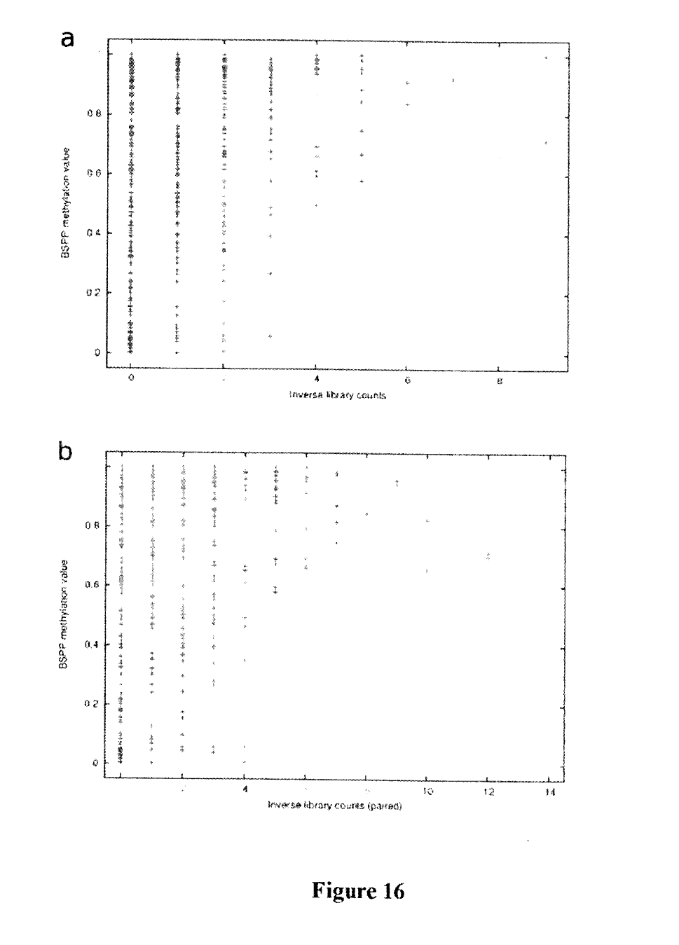

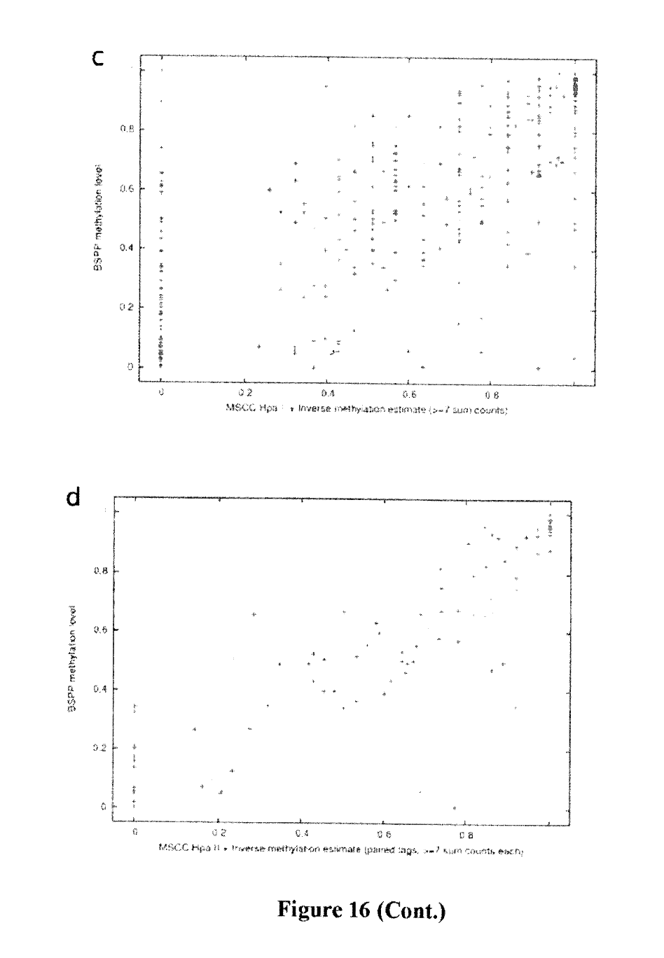

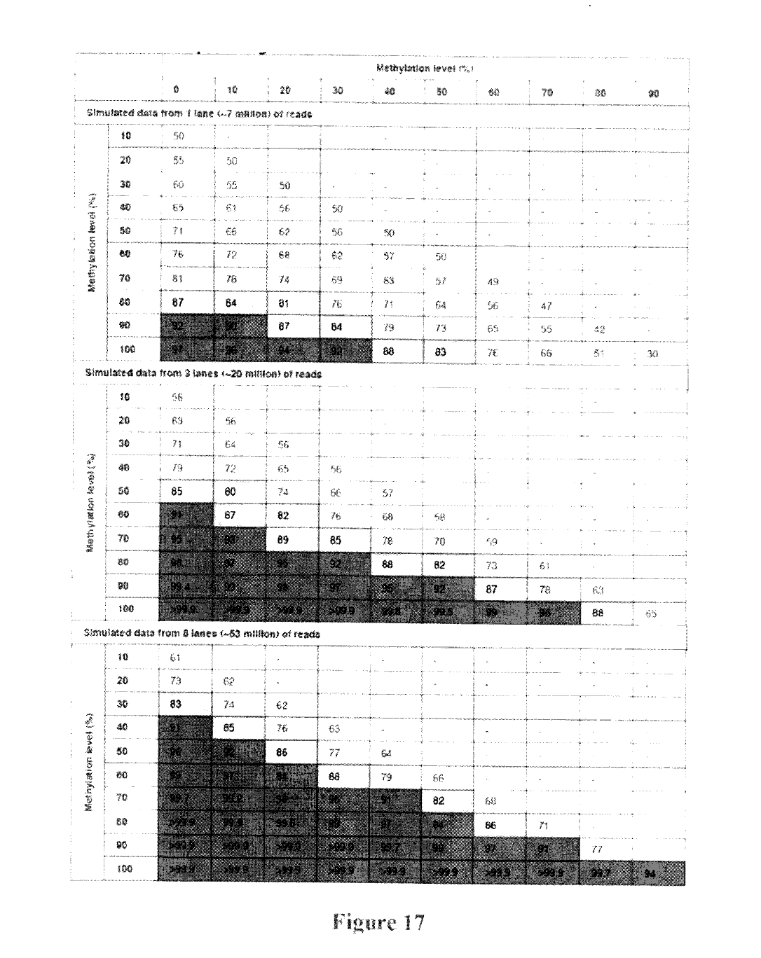

As a result, MSCC was more accurate at distinguishing moderately methylated sites from highly methylated sites than it was for distinguishing moderately from weakly methylated sites, although deeper sequencing coverage should improve accuracy (FIG. 17). In addition, preliminary data indicated that the accuracy could be improved by sequencing an ‘inverse library’ of methylated CCGG sites, which was constructed by dephosphorylating HpaII-digested fragment ends, digesting with MspI and then ligating an MmeI-containing adaptor to generate sequencing tags (FIG. 16). In the following analyses, however, only the MSCC HpaII data generated from three lanes of Illumina sequencing was utilized.

Compared to BSPP, which targeted several thousand data points covering approximately 400 genes, the MSCC technology covered the entire genome, allowing examination of the relationship between gene expression and cytosine methylation more thoroughly. Genes were split into five equal groups based on their expression levels and the running average of MSCC observations vs. gene position was plotted for each (FIGS. 2B-2D). A similar pattern of low promoter methylation and high gene body methylation was observed in high expression genes.

To investigate the amount of information contained within CpG islands, the data were separated into two groups according to whether a given site was located within or outside a CpG island (FIGS. 2E and 2F). Although methylation of CpG islands is known to suppress transcription, it was determined that, on average, sites outside CpG islands were responsible for most of the difference in methylation levels observed between the promoters of high and low expression genes.

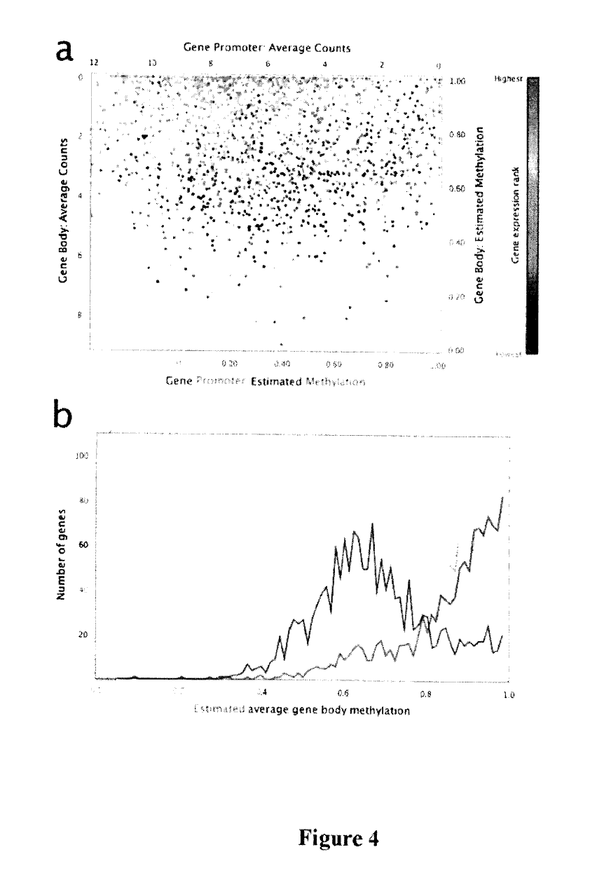

To explore how methylation information was correlated with gene expression on the level of individual genes, gene promoter methylation and gene body methylation of individual genes were compared. According to these two metrics, genes formed two clusters that corresponded to high and low expression levels (FIG. 4A). Gene body methylation appeared to be bimodally distributed, with two peaks corresponding to highly expressed and lowly expressed genes (FIG. 4B).

The rapid development of cheaper, massively parallel sequencing technologies (Khulan, B. et al., Comparative isoschizomer profiling of cytosine methylation: the HELP assay. Genome Res 16 (8), 1046-1055 (2006)) is opening the way for new strategies for studying biological processes (Schuster, S. C., Next-generation sequencing transforms today's biology. Nat Methods 5 (1), 16-18 (2008); Mardis, E. R., Next-generation DNA sequencing methods. Annu Rev Genomics Hum Genet 9, 387-402 (2008); Kahvejian, A., Quackenbush, J., & Thompson, J. F., What would you do if you could sequence everything? Nat Biotechnol 26 (10), 1125-1133 (2008)), including epigenetic features like DNA methylation (Meissner, A. et al., Genome-scale DNA methylation maps of pluripotent and differentiated cells. Nature 454 (7205), 766-770 (2008); Cokus, S. J. et al., Shotgun bisulphite sequencing of the Arabidopsis genome reveals DNA methylation patterning. Nature 452 (7184), 215-219 (2008); Lister, R. et al., Highly integrated single-base resolution maps of the epigenome in Arabidopsis. Cell 133 (3), 523-536 (2008)). BSPP and MSCC are two complementary methods that take advantage of the cheap, accurate and quantitative nature of new sequencing technologies to profile cytosine methylation at single-base resolution in targeted and genome-wide surveys.

### Example III

### Discussion

The data presented herein from both BSPP and MSCC methods shows a pattern of gene body methylation in the highly expressed genes of human cell lines. This is a phenomenon that has already been observed in Arabidopsis (Cokus, S. J. et al., Shotgun bisulphite sequencing of the Arabidopsis genome reveals DNA methylation patterning. Nature 452 (7184), 215-219 (2008); Lister, R. et al., Highly integrated single-base resolution maps of the epigenome in Arabidopsis. Cell 133 (3), 523-536 (2008); Zhang, X. et al., Genome-wide high-resolution mapping and functional analysis of DNA methylation in arabidopsis. Cell 126 (6), 1189-1201 (2006); Zilberman, D., Gehring, M., Tran, R. K., Ballinger, T., & Henikoff, S., Genome-wide analysis of Arabidopsis thaliana DNA methylation uncovers an interdependence between methylation and transcription. Nat Genet 39 (1), 61-69 (2007)), where it is associated with active genes. There is growing evidence in mammals: gene body methylation has been observed in the active human X chromosome when compared to the inactive X (Hellman, A. & Chess, A., Gene body-specific methylation on the active X chromosome. Science 315 (5815), 1141-1143 (2007)), and low methylation sites in the gene body have been associated with low expression genes in cancer cell lines (Shann, Y. J. et al., Genome-wide mapping and characterization of hypomethylated sites in human tissues and breast cancer cell lines. Genome Res 18 (5), 791-801 (2008)). Gene body methylation has been hypothesized to suppress spurious initiation of transcription within active genes in Arabidopsis (Zhang, X. et al., Genome-wide high-resolution mapping and functional analysis of DNA methylation in arabidopsis. Cell 126 (6), 1189-1201 (2006); Zilberman, D., Gehring, M., Tran, R. K., Ballinger, T., & Henikoff, S., Genome-wide analysis of Arabidopsis thaliana DNA methylation uncovers an interdependence between methylation and transcription. Nat Genet 39 (1), 61-69 (2007)) and a similar function may exist in mammals (Suzuki, M. M. & Bird, A., DNA methylation landscapes: provocative insights from epigenomics. Nat Rev Genet 9 (6), 465-476 (2008)).

In addition, it was determined that expression-related differences in promoter regions were much larger, on average, for CpGs outside islands. CpG islands and promoters have been the preferred target of many studies and have, in the past, guided the design of many methylation profiling experiments (Meissner, A. et al., Genome-scale DNA methylation maps of pluripotent and differentiated cells. Nature 454 (7205), 766-770 (2008); Illingworth, R. et al., A novel CpG island set identifies tissue-specific methylation at developmental gene loci. PLoS Biol 6 (1), e22 (2008); Weber, M. et al., Distribution, silencing potential and evolutionary impact of promoter DNA methylation in the human genome. Nat Genet 39 (4), 457-466 (2007); Bibikova, M. et al., High-throughput DNA methylation profiling using universal bead arrays. Genome Res 16 (3), 383-393 (2006)). In light the observations presented herein, less biased profiling methods are powerful in that they enable one of skill in the art the ability to discover aspects of methylation that might otherwise have been missed. As DNA sequencing costs drop, tools like BSPP and MSCC can be readily applied to study the epigenomic changes associated with developmental stages, environmental changes, disease states and the like.

### Example IV

Materials and Methods

Using approximately 30,000 padlock probes generated from Agilent's oligonucleotides

(FIG. 9A), three critical steps of technical development in the genotyping phase were tested (FIG. 9B). Although the protocol for allelic-specific extension and circularization (Step 8) had been well established (Hardenbol et al. (2003) Nat. Biotechnol. 21:673; Hardenbol et al. (2005) Genome Res. 15:269), two critical points were identified: 1) using Apyrase to remove contaminating nucleotides; and 2) adding polymerase and ligase after probes were annealed to the genomic templates) to ensure specific extension and ligation.

It was also determined that, due to the low ligation efficiency on genomic templates, amplification of circularized padlock probes by PCR (Step 10) was associated with high amplification biases. However, it was also determined that a pre-PCR Rolling Circle Amplification using either Bst polymerase or phi29 polymerase reduced the biases dramatically. The genotyping assay was verified using Sanger Sequencing, and it was confirmed that the genotyping assay is specific. In addition, it was determined that, in designing padlock probes, SNPs located within repetitive regions of the human genome should not be included, because the corresponding padlock probes tended be present at very high copy numbers after circularization and reduce the efficiency of genotyping assay.

### Cell Lines, Genomic DNA and RNA

Genomic DNA of GM06990 (a HapMap/ENCODE sample) was obtained from Coriell Cell Repository (Worldwide Website: ccr.coriell/org/Sections/Search/Sample_Detail.aspx?Ref=GM06990).

With the approval from Harvard Medical School's Institutional Review Boards (IRB), blood and skin biopsies were obtained from donors of the Personal Genome Project (PGP, Worldwide Website: personalgenomes.org). The EBV-transformed B-lymphocyte cell lines and the derivative genomic DNA for donors PGP1 (GM20431, Worldwide Website: ccr.coriell.org/Sections/Search/Sample_Detail.aspx?Ref=GM20431) and PGP9 (GM21833, Worldwide Website ccr.coriell.org/Sections/Search/Sample_Detail.aspx?Ref=GM21833) were generated and acquired from Coriell Cell Repository. Genomic DNA obtained directly from Coriell was used for methylation analysis of these lines, cultured cell lines were used for gene expression profiling. The primary fibroblast line for PGP9 was generated by and obtained from the Brigham Women's Hospital. The cultured cell line was used for both genomic DNA and gene expression profiling.

The iPS cell lines were derived by infecting primary human fibroblasts of PGP9 with highly concentrated retroviral OCT3, KLF4, SOX2 and c-MYC particles (Park, I. H. et al., Generation of human-induced pluripotent stem cells. Nat Protoc 3 (7), 1180 (2008)). The infected cells were trypsinized onto a feeder-layer after 4 days and maintained in hES media (KO-DMEM (Invitrogen), 20% KO-SR (Invitrogen), L-glutamine, non-essential amino acid, pen/strep, 55 μM 2-mercaptoethanol and 10 ng/ml bFGF). The iPS colonies were identified by their characteristic morphology after 3-4 weeks.

Immortalized lymphocytes were cultured in RPMI-1640 medium (Invitrogen) with 10% FBS (Invitrogen) and L-Glutamine (Invitrogen). Primary fibroblasts were cultured in DMEM/F12 medium with 15% FBS and 10 ng/μl EGF. Human iPS cell lines were grown on a feeder layer of mouse embryonic fibroblasts (Global Stem) in hES media, and mechanically separated from mouse cells prior to DNA/RNA extraction.

Genomic DNAs and total RNAs were extracted with AllPrep DNA/RNA/Protein Mini Kit (Qiagen).

RNA gene expression profiling for the PGP cell lines was done using Illumina's bead array technology (Worldwide Website: www.illumina.com/pages.ilmn?ID=5) through the service by Harvard Partner Center for Genetics and Genomics.

### Bisulfite Padlock Probe (BSPP) Design

A given set of padlock probes consists of two unique “arm” sequences that anchor a specific locus of interest, and a common center “backbone” sequence connecting two arms, the extension arm and the and ligation arm. The extension arm was the one where polymerization started from, and the ligation arm was the one where the polymerization ended and ligation occurred.

In the BSPP set, arms were up to 28 base pairs each in length, which was set to achieve a Tm range for specific capturing (see below). Probes were designed to target bisulfite-treated human DNA sequences chosen from the ENCODE regions. Potential probe locations were chosen according to the following criteria: first, a CpG dinucleotide existed at the 3′ end of the 10 base pair target, at the junction between the gap and ligation arm; second, CpG-free regions (for arm design) existed upstream and downstream of the 10 base pair span. The first criterion was chosen for two reasons: (1) the correct incorporation of CpG (or TpG if cytosine is unmethylated) depends on the high fidelity of both polymerase and ligase, and (2) the queried cytosine is positioned as close as possible to the sequencing primer in the final library molecules. Because the common backbone sequence was used as a sequencing primer, 28 bases of the arm sequence were sequenced before informative bases from the span were reached. Thus, there was a concern that the targeted CpG should be as close as possible in order to fall within the sequenced region and reduce low quality reads. Notably, because of the second criteria (no CpGs within the arms), the sites that were chosen from were actually biased against falling within CpG islands.

ENCODE region sequences were downloaded from UCSC in the form of Fasta files (Worldwide Website: genome.ucsc.edu/ENCODE/). All CpGs meeting these criteria were considered, and both strand possibilities were considered. Because there was concern about how well padlock probes would work with bisulfite-treated DNA, made no effort was made to target particular locations inside the ENCODE region. Instead, probes that seemed most optimal according to various criteria were chosen.

Each possible probe site was then subject to the following requirements: (A) arm length was chosen to fit a narrow melting temperature range of 50-55° C. (a Perl code to calculate melting temperature available upon request), (B) a minimum “G” content of 20% was required of both arms, (C) at least three non-CpG cytosines within the 15 bases of the “ligation” arm (to prevent probes targeting unconverted DNA).

Because bisulfite treated DNA loses a lot of sequence complexity as almost all cytosines become uracil and base-pair as thymines, there was concern about sequence uniqueness. This was addressed with steps D-F: (D) potential sites were selected based on average uniqueness for each arm, defined as the average frequency of all 15 base pair segments within it, (E) sites were selected based on an “internal uniqueness” score, the product of frequencies of the internal 15-mers on either side of the 10 base pair span, (F) potential mis-hybridization targets for remaining probes were removed by searching with BLAST against a custom “bisulfite treated” human genome (all non-CpG C's converted to T). Finally (step G), the set was trimmed to remove probes that overlapped in location to avoid hybridization conflicts.

Starting with 152,658 possible sites each step discarded the following percentage of probes:

| | |
|---|---|
| (A: Tm range) | 70% (45,282 remaining) |
| (B: G content) | 12% (39,675 remaining) |
| (C: non CpG C's) | 40% (23,020 remaining) |
| (D & E: uniqueness) | 24% (17,468 remaining) |
| (F: BLAST) | 36% (11,166 remaining) |
| (G: no overlap) | 14% (9,552 remaining) |

Because some probes contained more than one CpG within the 10 base pair span, this final set of 9,552 probes targeted 10,704 CpGs. It is noteworthy that the criteria chosen in this experiment targeting ENCODE regions were very stringent; accordingly these data not necessarily suggest that only a tiny fraction of CpG sites are targetable.

Bisulfite Padlock Probe Synthesis and Processing

To make the padlock probes amplifiable, 150-mer oligos having the following structure were designed. The common sequences (underlined) at both ends will be used to amplify the pool of synthesized oligos.

ACGGGTGGAAGATGGATGAT[ligation_28 nt]AGATCGGAAGAGCG
TCGTGTAGGGAAAGCTGAGCAAATGTTATCGAGGTC[extension_28
nt]GATCGTCCTTACACACTAGCCGTC

Using a programmable microarray (Agilent Technologies), a total of 9,552 such oligos (150-mers) were synthesized, which were cleaved off and collected in a single tube. The oligos were estimated to be about 0.18 fmol/species.

To amplify the oligos, real time PCR was performed on 1% of the synthesized oligos (1.8 amol/species, or ˜1 million molecules/species) to monitor the amplicon in a 100 μl reaction assembled with Platinum Taq supermix, 50 pmol each of primers (AP1_BS10.SS.U: A*C*G*GGTGGAAGATGGATGAU; *: phosphothioate modification, U: deoxyuridine), and AP2_BS10.p: /5phos/GACGGCTAGTGTGTAAGGAC), and 0.5× SYBR Green. The PCR program was 95° C. for 5 minutes, 15 cycles of 95° C. 30 seconds, 58° C. 1 minute, and 72° C. 1 minute, and finally 72° C. for 5 minutes. The PCR product was purified with Qiagen PCR purification kit and subsequently quantified. Using a 96-well plate, a 9.6 ml of PCR reaction was set up with 25 fmol of template along with Platinum Taq supermix, 4.8 nmol each of primers, and 0.5× SYBR Green. The same PCR program was used. The PCR products were purified by phenol:chloroform followed by Qiagen PCR purification kit, and a total of 37 μg of DNA was obtained.

The purified PCR product was split into eight reactions with 10 units of lambda exonuclease in 1× lambda exonuclease reaction buffer, and incubated at 37° C. for 45 minutes then 75° C. for 15 minutes. After being purified with QiaQuick columns, the ssDNA was quantified with Nanodrop to be 33 ng/μl in 200 μl total. This was split into four tubes, each of which was assembled with 50 μl of ssDNA (33 ng/μl), 6 μl of 10× DpnII reaction buffer, and 2 μl of 100 μM Guide DpnII BS10 (GGCTAGTGTGTAAGGACGATCNN). The “guide” oligo was annealed to the ssDNA by bringing the reaction to 95° C. for 5 minutes, followed by ramp to 60° C. at 0.1° C./sec, then 60° C. for 10 minutes, and 37° C. for 1 minute. In each tube, 5 μl of DpnII (10 u/μl) (NEB) and 5 μl of USER enzyme (1 u/μl) (NEB) were added and incubate at 37° C. for 3 hours. The final product was loaded into multiple lanes of 6% TBE Urea precast gels (Invitrogen), and the desired band was cut and subsequently purified. Finally, the concentration of padlock probes was quantified on a 6% TBE urea gel along with a quantitative low mass DNA ladder (Invitrogen). The probes were at 9 ng/μl, which is 257 nM (27 pM for each of 9,552 species).

### Bisulfite Treatment of Genomic DNA

Bisulfite treatment was performed using the EZ DNA Methylation-Gold Kit (Zymo

Research). The genomic DNA (2-10 μg) was split into multiple tubes with 500 ng each, and converted with sodium bisulfite according to manufacture's protocol. The typical yield was 50-75% after bisulfite conversion. The final product was eluted with dH₂O and concentrated to be >100 ng/μl.

### CpG Padlock Capturing and Construction of Sequencing Libraries

A 15 μl of reaction was set up using 1× Ampligase buffer (Epicentre), 1 μg (˜0.5 amol of haploid) of genomic DNA, and 33.5 ng (˜1 pmol) of probes. Using a thermal cycler, the reaction was denatured at 95° C. for 10 minutes, ramped to 64° C. for 5 hours, then 62° C. for 5 hours, and finally hybridized at 60° C. for 24 hours. At 60° C., the gap filling and sealing mix was added (0.5 pmol of dNTPs (USB), 2 units of Taq Stoffel fragment (Applied Biosystems), and 2.5 units of Ampligase (Epicentre) in Ampligase storage buffer (Epicentre)) totaling 2 μl, and incubated the reaction at 60° C. for 2 hours. The reaction was then cycled 5 times of 95° C. for 2 minutes and 60° C. for 5 hours. To digest the linear DNA, the incubation temperature was lowered to 37° C., and 2 μl of Exonuclease I (20 units/μl) (USB) and 2 μl of Exonuclease III (200 units/μl) (USB) was immediately added. The reaction was incubated at 37° C. for 2 hours followed by 94° C. for 5 minutes.

To amplify the circularized padlock probes, for each sample two 100 μl reactions were set up, each of which was assembled with 50 μl of 2× iQ SYBR Green supermix (Bio-Rad), 10 μl of template from above, 40 pmol each of primers (CIR_for_SLXA: CAAGCAGAAGACGGCATACGAGCTCTGAGCAAATGTTATCGAGGTC and CIR_rev_SLXA: AATGATACGGCGACCACCGACACTCTTTCCCTACACGACGCTC), and water. The reactions were carried out on a real time PCR instrument to avoid over-amplification by monitoring the amplicons. The PCR program was 96° C. for 3 minutes, 5 cycles of 96° C. for 15 seconds, 60° C. for 30 seconds, and 72° C. for 30 seconds, then 13 cycles of 96° C. for 15 seconds, and 72° C. for 1 minute, and finally 72° C. for 5 minutes. A small fraction (-2%) of PCR product was run on a TBE polyacrylamide gel to check if the desired bands were present (e.g., FIG. 5A), and if so, the rest was loaded and the desired band was cut from the gel. To purify the DNA from the polyacrylamide gel, 3× volume of TE (pH 8.0) was added followed by incubation at 60° C. for at least 30 minutes. Q 0.2 μm Nanosep spin column (Pall) was used to remove gel fragments and used ethanol precipitation to recover the DNA from the solution, and finally resuspended with 30 μl dH₂O.

### BSPP Sanger Sequencing Validation

To validate the accuracy of the methylation level determined by the padlock capturing and Illumina Genome Analyzer sequencing, primers for 33 targeted sites were designed (Table 3) and PCR amplification followed by conventional Sanger sequencing were performed. For each 10% interval of methylation level (0%, 10%, 20%, . . . 90%, and 100%), three sites were randomly chosen (thus 33 sites total). For each site, real time PCR was performed in duplicates of 40 μl reaction with 50 ng of bisulfite converted GM06990 genomic DNA, 1× iQ SYBR Green supermix (Bio-Rad), and 500 nM each of forward and reverse primers. The PCR program was 96° C. for 3 minutes, 40 cycles of 95° C. for 30 seconds, 62° C. for 1 minute, and 72° C. for 1 minute, and finally 72° C. for 5 minutes.

TABLE 3
PCR primers used to validate methylation level determined by BSPP. This is a
table of the targets and primers that were used for Sanger sequencing validation of the
BSPP data.

| | | Methylation | |
|---|---|---|---|
| | Location of | level | |
| | queried cytosine | measured | |
| Primer name | (chr_position) | by BSPP | Primer sequence |
| 0.0_F_chr5_141931200 | chr5_141931200 | 0% | TTAAAGGATTTTAGGAATTTTATTAGTT |
| 0.0_R_chr5_141931200 | chr5_141931200 | 0% | AAATACTATCAAAAACTACTTCCAAAC |
| 0.0_F_chr11_2278418 | chr11_2278418 | 0% | GTTGTGGTTAGATTTGGTTTTT |
| 0.0_R_chr11_2278418 | chr11_2278418 | 0% | ACCTTAACCTCCCTAAAACTAATAA |
| 0.0_F_chr21_32895366 | chr21_32895366 | 0% | AAGTTTTTTTAGTAAGGTTGGGA |
| 0.0_R_chr21_32895366 | chr21_32895366 | 0% | CACTACACTCTATCCTAAACAACAA |
| 0.1_F_chr5_131430745 | chr5_131430745 | 10% | ATTTTTTGGTTTTAGGTTTATAGTG |
| 0.1_R_chr5_131430745 | chr5_131430745 | 10% | AAATCTCTCTCAAAAATTCCTTAA |
| 0.1_F_chr11_4861886 | chr11_4861886 | 10% | TTAATTTGGTTTGTTGATTTTAGTT |
| 0.1_R_chr11_4861886 | chr11_4861886 | 10% | CTCACCTAAAAAATATATAAATCCC |
| 0.1_F_chr22_31384238 | chr22_31384238 | 10% | GTGAATAGGTTAAGTGAGGTAGAAG |
| 0.1_R_chr22_31384238 | chr22_31384238 | 10% | AAAAAAATCAAACACCAACTATAAA |
| 0.2_F_chr11_2136827 | chr11_2136827 | 20% | GGGTGAGTAGTAGGTTTGTAGTAAA |
| 0.2_R_chr11_2136827 | chr11_2136827 | 20% | CAAATAACACCATAAACTAAAACAA |
| 0.2_F_chr14_98615277 | chr14_98615277 | 20% | TTTGTTTTAAGTTTTTAAAGGGTAA |
| 0.2_R_chr14_98615277 | chr14_98615277 | 20% | AAATACTCTAAATTTCTCACAACCTAC |
| 0.2_F_chr19_59719262 | chr19_59719262 | 20% | GTAGGTTTTAGGAATTTTAGGATAGA |
| 0.2_R_chr19_59719262 | chr19_59719262 | 20% | TAAAACCCTTTACATTTCAATAAAT |
| 0.3_F_chr2_220372916 | chr2_220372916 | 30% | TTTTATTTAGAGTTGTTTTATGTTAAGG |
| 0.3_R_chr2_220372916 | chr2_220372916 | 30% | ATCTCCTATAAATCCCCAATTAATA |
| 0.3_F_chr5_131431205 | chr5_131431205 | 30% | GTTTTGGTAGAGATTTGTTTGG |
| 0.3_R_chr5_131431205 | chr5_131431205 | 30% | AAAAAAAACCCCTACTCTACTACTC |
| 0.3_F_chr11_64232560 | chr11_64232560 | 30% | AGGTGATATGAGGAAGTATTGTTAT |
| 0.3_R_chr11_64232560 | chr11_64232560 | 30% | AAACCTCCATACTAAAAAATTTACAT |
| 0.4_F_chr5_141987439 | chr5_141987439 | 40% | TTAGATTTTATTTTGGATTTTGAAA |
| 0.4_R_chr5_141987439 | chr5_141987439 | 40% | CTCTACAAAAACTTAACCCTTAAAA |
| 0.4_F_chr16_25811080 | chr16_25811080 | 40% | GAAAATTTGATTTTAAAAGAATGTG |
| 0.4_R_chr16_25811080 | chr16_25811080 | 40% | TTTTAAAAATAACAAAATCAACTCC |
| 0.4_F_chr22_31195396 | chr22_31195396 | 40% | TTAATTGAAGATTAAATATTTTTGAGAT |
| 0.4_R_chr22_31195396 | chr22_31195396 | 40% | CTTTAAAATTTCCTTTTAACCAAAT |
| 0.5_F_chr7_27107421 | chr7_27107421 | 50% | GGAGTTTTTAAGGTTTTTATATTTTTT |
| 0.5_R_chr7_27107421 | chr7_27107421 | 50% | CCAACACACAACTTCTAAAACTAA |
| 0.5_F_chr11_1943248 | chr11_1943248 | 50% | TTAGGAGGTGTTTAGATGATTTTAG |
| 0.5_R_chr11_1943248 | chr11_1943248 | 50% | CCCAATATATACACAACCAAAAC |
| 0.5_F_chr11_130700083 | chr11_130700083 | 50% | ATGTTTGTGAAAGTAGGAGTTTATT |
| 0.5_R_chr11_130700083 | chr11_130700083 | 50% | TACTCTTATCCCTTCTCCCTAATAT |
| 0.6_F_chr5_131557355 | chr5_131557355 | 60% | GATTGTTAGTATTGTAGAGGGTTTG |
| 0.6_R_chr5_131557355 | chr5_131557355 | 60% | AACTTCAATAATACATTAAAATAAAATTTT |
| 0.6_F_chr16_25856798 | chr16_25856798 | 60% | GATTTTTAGTTTTGTAGTGTTGAGG |
| 0.6_R_chr16_25856798 | chr16_25856798 | 60% | CTAATAAAATCTAAATTCAAAAACACTTAT |
| 0.6_F_chrX_153233491 | chrX_153233491 | 60% | TTTGTGTTAGTTTTGGGTTTAATAT |
| 0.6_R_chrX_153233491 | chrX_153233491 | 60% | CAACCTTCAATAAAAACAAACTATT |
| 0.7_F_chr1_149600103 | chr1_149600103 | 70% | TAAGTTAGGTGTTGGGAGTTAATAG |
| 0.7_R_chr1_149600103 | chr1_149600103 | 70% | TAAAATATCCACCTCAACTAAAATC |
| 0.7_F_chr11_64054275 | chr11_64054275 | 70% | TGATTTTATTTTGAAAGTGAAGTTT |
| 0.7_R_chr11_64054275 | chr11_64054275 | 70% | ATTTTCACAAAAACTATAAAACACAA |
| 0.7_F_chrX_153373129 | chrX_153373129 | 70% | GATTTGTTTGTTTTTTTAAATTTTG |
| 0.7_R_chrX_153373129 | chrX_153373129 | 70% | AAATTAATTCCAATTACACCAATAA |
| 0.8_F_chr21_39672131 | chr21_39672131 | 80% | AAAATATTGGGATTATAGGTATGAGT |
| 0.8_R_chr21_39672131 | chr21_39672131 | 80% | AACTTCTAAACTAACCAAAACAAAA |
| 0.8_F_chr22_31794899 | chr22_31794899 | 80% | TGTTTTAGGAGGTGAATAAATTAAT |
| 0.8_R_chr22_31794899 | chr22_31794899 | 80% | AACCTTATAAACTTCACAATCAAAC |
| 0.8_F_chrX_152958511 | chrX_152958511 | 80% | TTTATTTAATATATGTTGGATGAATAATTA |
| 0.8_R_chrX_152958511 | chrX_152958511 | 80% | CTAAAACCCTCCTCAATAACTTC |
| 0.9_F_chr6_108430751 | chr6_108430751 | 90% | TGTTAATGAATATAATGTTTTGTTTTT |
| 0.9_R_chr6_108430751 | chr6_108430751 | 90% | TAATACCCAACTAACTCCCTACTAA |
| 0.9_F_chr8_119031762 | chr8_119031762 | 90% | TTATAGTTTGGGTGATAGAGTAAGATT |
| 0.9_R_chr8_119031762 | chr8_119031762 | 90% | AAACCCTAAACAAAATACTCAATATAA |
| 0.9_F_chr22_30304462 | chr22_30304462 | 90% | GGTAGATATGTTGTTGTGTGTAGAA |
| 0.9_R_chr22_30304462 | chr22_30304462 | 90% | AAAAAAACTTCATAACCAAAACTC |
| 1.0_F_chr2_118425897 | chr2_118425897 | 100% | TATGATAGAGGTGGTAGTAGAGGTG |
| 1.0_R_chr2_118425897 | chr2_118425897 | 100% | TTCCAATTATCTCCTAAACAAAATA |
| 1.0_F_chr6_74157666 | chr6_74157666 | 100% | AAAAGTTTAGTATATTTTGTGGTTTTT |
| 1.0_R_chr6_74157666 | chr6_74157666 | 100% | CACCAATATATTATAAAAAAACTCTTTATT |
| 1.0_F_chr11_1933957 | chr11_1933957 | 100% | GGGGTAGATATTAGGTTTTAAAGAG |
| 1.0_R_chr11_1933957 | chr11_1933957 | 100% | AACTACAAAAACTCCTCAACAAA |

Rather than isolating and sequencing many clones for each target, Sanger sequencing was performed on the raw PCR products (an average of three sequencing reaction per site) and methylation levels were inferred based on the sequence traces using the program PeakPicker (Ge, B. et al., Survey of allelic expression using EST mining. Genome Res 15 (11), 1584 (2005)) to measure the heights of a set of peaks within the trace. However, due to the lack of cytosines in the sequence, the cytosine in the queried CpG had an abnormal peak height, which made comparison between C and T peaks not informative. Instead, he height of the “T” peak within the CpG location as well as the heights of four upstream and downstream “T” peaks were measured. The surrounding peaks provided normalization for measuring the relative height of the target “T” peak to a 100% value. The ratio of the target “T” (in the CpG) to the average of the surrounding “T” peaks was inferred to reflect the fraction of unmethylated cytosine at that position. This was similar to the principle applied in the commercially available software ESME (Lewin, J. et al., Quantitative DNA methylation analysis based on four-dye trace data from direct sequencing of PCR amplificates. Bioinformatics 20 (17), 3005 (2004)). Because sequencing reactions were performed for multiple times and from both directions, these generated multiple estimates from which average and standard deviation values were obtained for the methylation of that site (FIG. 1C).

### BSPP Library Sequencing, Placement of Reads, and Determination of Methylation

Libraries were sequenced using a single lane of Illumina's Solexa sequencing system per sample (Table 4). The sequencing primer used was CACTCTTTCCCTACACGAC GCTCTTCCGATCT. Reads were mapped by using BLAST with a custom database of the 9,552 target sequences (with an “N” at any CpG cytosine position). Any placements with mismatches in the 10 base pair “span” (bases polymerized rather than originally part of the probe) were discarded to reduce the chance of including data from probes mis-hybridized to other genomic locations. Accepted reads were then combined for each given probe to determine methylation at each position, based on the number of reads that had a C or T at a given position. Only probes with at least 10 reads in a sample were used to measure methylation level.

TABLE 4
BSPP Illumina sequencing statistics. This table contains statistics for
the number of reads and number of matched reads for the Illumina runs
used for the BSPP method. Each sample (row) corresponds to a single
lane of sequencing.

| | | | | Number of | Number of |
|---|---|---|---|---|---|
| | | Number | Number | probes with at | probes with at |
| | Number | matched | accepted | least 1 read | least 10 reads |
| Sample | of reads | (percentage) | (percentage) | (percentage) | (percentage) |
| GM06990 | 4,107,685 | 3,689,651 | 2,040,725 | 7,453 (78.0%) | 5,833 (61.1%) |
| Tech rep 1 | | (89.8%) | (49.7%) | | |
| GM06990 | 3,015,101 | 2,794,275 | 2,259,755 | 7,418 (77.7%) | 5,952 (62.3%) |
| Tech rep 2 | | (92.7%) | (74.9%) | | |
| PGP1 | 2,683,213 | 2,487,042 | 1,900,742 | 8,079 (84.6%) | 6,754 (70.7%) |
| lymphocyte | | (92.7%) | (70.8%) | | |
| PGP9 | 8,668,249 | 7,978,035 | 5,807,123 | 8,109 (84.9%) | 7,195 (75.3%) |
| lymphocyte | | (92.0%) | (67.0%) | | |
| PGP1 | 1,468,378 | 1,364,329 | 1,101,446 | 7,131 (74.7%) | 5,384 (56.4%) |
| fibroblast | | (92.9%) | (75.0%) | | |
| PGP9 | 3,242,845 | 2,921,455 | 2,214,021 | 7,865 (82.3%) | 6,630 (69.4%) |
| fibroblast | | (90.1%) | (68.3%) | | |
| PGP1 iPS | 283,724 | 247,492 | 193,621 | 6,566 (68.7%) | 3,942 (41.3%) |
| | | (87.2%) | (68.2%) | | |
| PGP9 iPS | 528,421 | 478,597 | 369,861 | 7,061 (73.9%) | 4,790 (50.1%) |
| clone 1 | | (90.6%) | (70.0%) | | |
| PGP9 iPS | 8,973,759 | 8,281,843 | 5,800,724 | 8,507 (89.1%) | 7,606 (79.6%) |
| clone 2 | | (92.3%) | (64.6%) | | |

### Comparison of GM06990 Methylation Levels and Gene Expression Levels

To compare our methylation data for the GM06990 cell line to gene expression levels, the Affymetrix PolyA+ RNA signal track was downloaded from UCSC (http://genome.ucsc.edu/ENCODE/). The data were examined for exons as annotated by RefGene, and the median value was determined as a record of gene expression level (for multiple possible transcripts, the transcript with the smallest difference between median values in the exons was taken). After excluding genes on the X chromosome, an expression ranking for 347 genes was obtained.

Each methylation data point was assigned position information according to its location relative to nearby genes. To create a profile examining methylation over an entire gene, sites within a gene were given a position value based on their relative position within the gene from transcriptional start to end (a fraction between 0 and 1). Sites upstream or downstream of genes were recorded according to number of bases from the gene boundaries. Genes were split into two groups based on expression level and for each group the data was combined to create a methylation profile by calculating the running median (and quartiles) using a window of 0.1 inside the gene and 3000 base pairs outside.

### Comparison of GM06990 Methylation and Chromatin Immunoprecipitation (ChIP) Data

To compare cytosine methylation levels with histone modifications, the Sanger ChIP data for the GM06990 cell line was downloaded from the ENCODE project data at UCSC (http://genome.ucsc.edu/ENCODE/). The raw ChIP score was plotted for a given experiment against the methylation observed at that position to look for correlation(s) between cytosine methylation and histone modification types.

Rather than seeing the expected correlation with H3K4 methylation, correlations with histone modifications (H4ac, H3K27me3, H3K29me3) were observed that had significant correlation with gene expression over the bodies of genes. Without intending to be bound by scientific theory, this is probably because few of the sites were close to the transcription start sites of genes (5.5% within 1 kb) but most were distributed over the bodies of genes (55.1%). To visualize the profiles of histone modification over the body of genes, the Sanger ChIP data was used to create running medians and quartiles for the high and low expression gene sets analogous to the one created for our cytosine methylation data.

### PGP Cell Line Methylation Analysis

The methylation patterns of different cell lines were evaluated by comparing methylation levels at all locations for which data was obtained from both lines (FIG. 9). For the PGP cell lines, gene expression described herein was used, and the genes were ranked according to expression level. The genes were then split into two groups as before. Running median and quartile profiles were created as with GM06990 data (FIG. 10).

It was observed that PGP9 B-cells showed higher overall methylation compared to PGP1 B cells. Without intending to be bound by scientific theory, this may have been an artifact of cell culture differences (an increasing number of passages in cell culture of neural precursor cells has, for example, been observed to result in gradual hyper-methylation (Meissner, A. et al., Genome-scale DNA methylation maps of pluripotent and differentiated cells. Nature 454 (7205), 766 (2008))).

### Methyl Sensitive Cut Counting (MSCC) Library Creation

Methyl sensitive cut counting uses a library created from all ends produced by HpaII digestion. To create the two adapters needed in the library construction, the following PAGE purified oligos were ordered from Integrated DNA Technologies (IDT).

AdA:
CAAGCAGAAGACGGCATACGAAGAGTCTCTATATGCATCGATGCAGATCA
CGATCCGA
AdA_RC:
CGTCGGATCGTGATCTGCATCGATGCATATAGAGACTCTTCGTATGCCGT
CTTCTGCTTG
AdB:
AATGATACGGCGACCACCGAGATCTACACTCTTTCCCTACACGACGCTCT
TCCGATCTNN
AdB_RC:
AGATCGGAAGAGCGTCGTGTAGGGAAAGAGTGTAGATCTCGGTGGTCGCC
GTATCATT

These were resuspended with TE 8.0 to a concentration of 40 μM. Equal amounts of AdA and AdA_RC were mixed to form a 20 μM solution of adapter A, and equal amounts of AdB and AdB RC were mixed to form a 20 μM solution of adapter B. To hybridize, these were placed in a PCR machine programmed to hold at 94° C. for 5 minutes, then drop 0.1° C. every 2 seconds, finally holding at 4° C. The resulting adapters had this design:

Adapter A
5′ CAAGCAGAAGACGGCATACGAAGAGTCTCTATATGCATCGATGCAGATCACGATCCGA 3′
3′ GTTCGTCTTCTGCCGTATGCTTCTCAGAGATATACGTAGCTACGTCTAGTGCTAGGCTGC 5′
Adapter B
5′ AGATCGGAAGAGCGTCGTGTAGGGAAAGAGTGTAGATCTCGGTGGTCGCCGTATCATT 3′
3′ NNTCTAGCCTTCTCGCAGCACATCCCTTTCTCACATCTAGAGCCACCAGCGGCATAGTAA 5′

Adapter A contained: Solexa bridge PCR sequence (CAAGCAGAAG ACGGCATACGA), 5′ CG overhang that matches HpaII cut ends (underlined GC), MmeI recognition site (bold text, complete after ligation). Adapter B contained: Solexa bridge PCR sequence (AGCGGCATAGTAA), Solexa genomic DNA sequencing primer (TCTAGCCTTCTCGCAGCACATCCCTTTCTCACA), 5′ NN overhang that matches MmeI cut ends. Final library molecules will have a length of 137-138 base pairs and look like this:

CAAGCAGAAGACGGCATACGAAGAGTCTCTATATGCATCGATGCAGATCAC
GAGTTCGTCTTCTGCCGTATGCTTCTCAGAGATATACGTAGCTACGTCTAG
TGCTTCCGACGGNNNNNNNNNNNNNNNNNNAGATCGGAAGAGCGTCGTGTA
GGGAAAGAGTGAGGCTGCCNNNNNNNNNNNNNNNNNNTCTAGCCTTCTCGC
AGCACATCCCTTTCTCACTAGATCTCGGTGGTCGCCGTATCATTATCTAG
AGCCACCAGCGGCATAGTAA

To create an MSCC HpaII library, 2 μg of genomic DNA was used to assemble a 100 μl reaction with 20 units of HpaII (NEB) in 1× NEBuffer 1. This solution was incubated at 37° C. for 2 hours and then 65° C. for 20 minutes. 1.66 μl of 10 μM adapter A was a to the mixture, along with 12 μl 10 mM ATP (NEB) and 6 μl T4 DNA ligase (NEB). This concentration of adapter was estimated to be 3.3-fold excess to cut ends if all possible HpaII targets were cut (but, because most sites are methylated, it should actually be in the range of 8-to-15-fold excess). The ligation mixture was incubated at 16° C. for 4 hours, then at 65° C. for 15 minutes. After ligation, ethanol precipitation was performed by adding 2 μl NF pellet paint (Novagen), 14 μl 3M sodium acetate, 280 μl ethanol, left overnight at −20° C. The precipitate was spun down, washed with 75% ethanol, and allowed to air dry. Because the adapters were not phosphorylated (preventing self ligation), only one of the two backbones of the adapters was ligated. To perform a nick repair, the pellets were resuspended to assemble a 50 μl reaction mixture with 8 units Bst DNA polymerase large fragment (NEB), 200 μM dNTP concentration and 1× thermopol buffer (NEB). This mixture was incubated at 50° C. for 20 minutes, then at 85° C. for 25 minutes. The nick-repaired fragments were precipitated by adding 6 μl 3M sodium acetate and 120 μl ethanol and storing at −20° C. for several hours. MmeI digestion was then performed by resuspending the pellets into a 50 μl reaction mixture containing 2 units of MmeI (NEB), 50 μM SAM (NEB) and 1× NEBuffer 4. This was incubated at 37° C. for 2 hours, then 80° C. for 20 minutes. 1.66 μl of 10 μM adapter B was add the mixture, along with 6.1 μl 10 mM ATP (NEB) and 3 μl T4 DNA ligase (NEB), then incubated at 16° C. for 4 hours, and 65° C. for 15 minutes.

To purify the target product before PCR amplification, the resulting unpurified reaction mixture was run on a 6% non-denaturing TBE polyacrylamide gel (NOVEX), alongside a NEB Low Molecular Weight DNA ladder. This size range was cut from the gel using SYBR Gold stain (Invitrogen) and Dark Reader (Clare Chemical) to avoid UV exposure. DNA was eluted from the polyacrylamide gel by incubating gel fragments in 2× volume of TE 8.0 and shaking at 60° C. for 30 minutes. A 0.2 μm Nanosep spin column (Pall) was used to remove gel fragments and used ethanol precipitation (with 2 μl NF Pellet Paint) to recover the DNA from the solution, and finally resuspended with 30 μl TE 8.0.

Because the concentration of this library could have been too low for sequencing, 25 μl of the sample was amplified by assembling a 100 μl PCR mixture containing 500 nM each of primers (SLXPCR-AdA: CAAGCAGAAGACGGCATACGA and SLXPCR-AdB: AATGATACGGCGACCACCGAG), 200 μM dNTPs, 1× HF buffer and 2 units iProof (Bio-Rad). This mixture was then denatured at 98° C. for 30 seconds, cycled 8 times 98° C.-10s/67° C.-15s/72° C.-15s, then held at 72° C. for 5 minutes. The resulting product was purified with the QiaQuick PCR clean-up kit (Qiagen), eluting with 30 μl EB. Sample concentration was measured with a Nanodrop, then diluted to 840 pg/μl (10 nM) for Illumina/Solexa sequencing.

The MspI control library was constructed in the same manner as the HpaII library, with the following changes: (i) in the first step 40 units of MspI (NEB) were used in place of HpaII and NEBuffer 2 was used instead of NEBuffer 1; and (ii) no amplification was done after gel purification.

The inverse library was constructed in this manner: HpaII digestion was performed as done in the HpaII library. After this, 10 units Antarctic Phosphatase (NEB) and 11 ml 10x Antarctic Phosphatase Buffer (NEB) were added to the mixture, which was then incubated at 37° C. for 1 hour, and 65° C. for 15 minutes. DNA was purified with phenol:chloroform followed by ethanol precipitation. The DNA was then resuspended and treated in the same manner as the MspI control library.

MSCC Sequencing and Placement of Reads

In total, three lanes of sequencing were performed, two lanes for technical replicate 1 and one lane for replicate 2 (Table 5).

TABLE 5
MSCC Illumina sequencing statistics. This table contains statistics for
the number of reads and number of matched reads for the Illumina runs
used for the MSCC method. Each row corresponds to a single
lane of sequencing.

| | | Number | Number | Percentage | Average |
|---|---|---|---|---|---|
| | Number of | matched | accepted | of tags seen | number of reads |
| Sample | reads | (percentage) | (percentage) | at least once | per tag |
| PGP1L | 6,052,886 | 3,598,311 | 1,765,709 | 38.0% | 0.77 |
| HpaII | | (59.4%) | (29.2%) | | |
| Tech rep 1, | | | | | |
| round 1 | | | | | |
| PGP1L | 5,759,738 | 4,233,294 | 2,303,336 | 43.4% | 1.0 |
| HpaII | | (73.5%) | (40.0%) | | |
| Tech rep 1, | | | | | |
| round 2 | | | | | |
| PGP1L | 8,579,795 | 6,397,139 | 3,536,353 | 53.6% | 1.5 |
| HpaII | | (74.6%) | (41.2%) | | |
| Tech rep 2 | | | | | |
| PGP1L | 20,392,419 | 14,228,744 | 7,605,398 | 65.7% | 3.3 |
| HpaII | | (69.8%) | (37.3%) | | |
| total | | | | | |
| PGP1L | 10,423,134 | 8,682,641 | 4,319,599 | 76.0% | 1.9 |
| MspI | | (83.3%) | (41.4%) | | |
| Control | | | | | |
| PGP1L | 6,355,775 | 4,954,057 | 2,172,381 | 45.3% | 0.94 |
| Inverse | | (77.9%) | (34.2%) | | |
| library | | | | | |

Using the human genome reference sequence, a list of all possible HpaII sites was created. Each CCGG site present generated two possible tags, from upstream and downstream sequence. Statistics for possible HpaII sites, and other enzymes, are available in Table 6. Reads were matched (using an in-house Perl program) if they were within two bases of an expected tag. A read was then accepted if it met two criteria: (a) it was either exact and there were no single mismatch possibilities, or it had a single mismatch and there were no double mismatch possibilities, and (b) it matched a subset of “unique” positions that were more than one base different in sequence from all other tags (1.4 million locations). Note that the amount of information and accuracy obtained for each site was not related to “coverage” of the site. Sites with no reads were inferred to be highly methylated (and hence uncut and unrepresented in the library). Sites with more reads were inferred to have lower methylation. Read counts information for all unique HpaII sites are set forth in Table 7.

TABLE 6
Methylation sensitive enzymes and their site
frequencies. This table contains some statistics
for HpaII and some other methylation-sensitive
enzymes: the number of sites and number of
“unique” sites that could be profiled by MSCC.
Based on the March 2006 human reference sequence
(NCBI Build 36.1) downloaded from UCSC. Unique
sites were based on tags created with MmeI (18
or 19 bases of sequence) and were required to be
at least two bases different from all other
possible tag sequences. Numbers of unique sites
for enzymes other than HpaII were estimates
based on analysis of a random set of 10,000
locations.

| | | Number of | Number of |
|---|---|---|---|
| Restriction | Recognition | sites in | “unique” |
| enzyme | site | human genome | sites |
| HpaII | CCGG | 2,321,216 | 1,417,432 |
| | | | (61.1%) |
| HhaI, CfoI | GCGC | 1,674,129 | ~950,000 |
| | | | (60%) |
| AciI | CCGC | 4,153,824 | ~2,500,000 |
| | | | (60%) |
| HpyCH4IV, | ACGT | 2,167,347 | ~1,500,000 |
| MaeII | | | (70%) |
| BstUI, MvnI | CGCG | 693,643 | ~420,000 |
| | | | (60%) |

TABLE 7
MSCC data (separate file) and description of columns. This Table
contains the locations and read counts data for all unique HpaII
sites profiled with MSCC. Each site could produce two possible
tags: “strand” refers to the two strands based on whether
they are generated from upstream (minus) or downstream (plus)
sequence. Although the read counts are separated here, the MSCC
data analysis used the sum of columns 4, 5 and 6.

| Column | Description |
|---|---|
| 1 | chromosome |
| 2 | location |
| 3 | strand |
| 4 | HpaII Technical replicate 1, sequencing lane 1 |
| 5 | HpaII Technical replicate 1, sequencing lane 2 |
| 6 | HpaII Technical replicate 2 |
| 7 | MspI control |
| 8 | Inverse library |

Based on a median over-dispersion of 62% compared to Poisson standard deviations when data was binned, HpaII tag counts were modeled as arising from gamma-Poisson and simulated 1, 3 and 8 lanes of read count data (FIG. 17). Paired tags were then modeled as a sum of two independent numbers generated from the same distribution. Using this model, given 1, 3 and 8 lanes of sequencing data, it was estimated that the probabilities (shown in Table 8 with unit %) in observing more counts at one paired tag site compared to another paired tag site with higher underlying methylation level (note that the count number is anti-correlated with methylation level).

### Comparison of Methylation Level Determined by MSCC or BSPP

Because the same sample (PGP1 lymphocyte) was analyzed with the BSPP ENCODE set, these data sets overlap at 381 sites (345 of these have two unique tags, and so there is data for a total of 726 tags). The raw data was compared as the number of counts (MSCC combined data) vs. methylation level (BSPP data) for each point. It was expected, when data was averaged over many points, that a linear correlation between average number of counts and the level of methylation would be observed. For example, a site that was completely cut (0% methylation) should produce twice as many library molecules (and, on average, have twice as many observations) compared to a site that was 50% cut (50% methylation). Sites that were completely methylated were expected to have zero observations. The expected relationship between methylation level and the average counts observed for that methylation level was: methylation=1−C*average counts.

The 726 data points were divided into 22 equally sized groups of 33 data points. For each, the average methylation was calculated and the averages were used to determine the best fit value for C: 0.1128 (completely unmethylated sites have an average of 8.9 observations). The standard deviation was also determined and included for a Poisson distribution with a lambda predicted by the linear equation (FIG. 3B). Thus, the equation that was later used to relate average counts to methylation was: methylation=1−0.1128*average counts.

Comparison of MSCC Methylation with Gene Expression Levels

Gene positions calculated were based on the RefGene list downloaded from UCSC (for genes with multiple possible starts/ends, only the first entry was used). Using the same expression data as used earlier for the PGP1 lymphocyte cell line, a list of 17,546 genes was split into five equally sized groups based on their gene expression levels.

As with the BSPP data, the position of each CpG position was recorded relative to the gene start, the gene end, and the fraction within the gene. A running average of counts was created for each. FIG. 2B used a window of 5000 data points, and FIGS. 2C-2F used a window of 2000 data points or 200 base pairs (the larger of the two; for each step an average position as well as average methylation was taken for all the data points). For FIG. 2B, upstream and downstream methylation were combined with fractional gene position to create an “average gene.” For FIGS. 2C and 2D, positions relative to 5′ and 3′ ends of the gene were used, respectively, to create average methylation profiles for each end of genes. For FIGS. 2E and 2F, the MSCC data was split into two groups based on whether the site profiled was within a CpG island (based on UCSC's CpG island annotation). The data from these two groups was then used to generate profiles of promoter methylation based only on that subset of data (inside CpG islands and outside CpG islands, respectively). Counts were normalized for local CpG density (surrounding 200 base pairs), for MspI control library counts, and, for the in-gene in FIG. 2B, for gene length.

### MSCC Methylation Profiles for Individual Genes

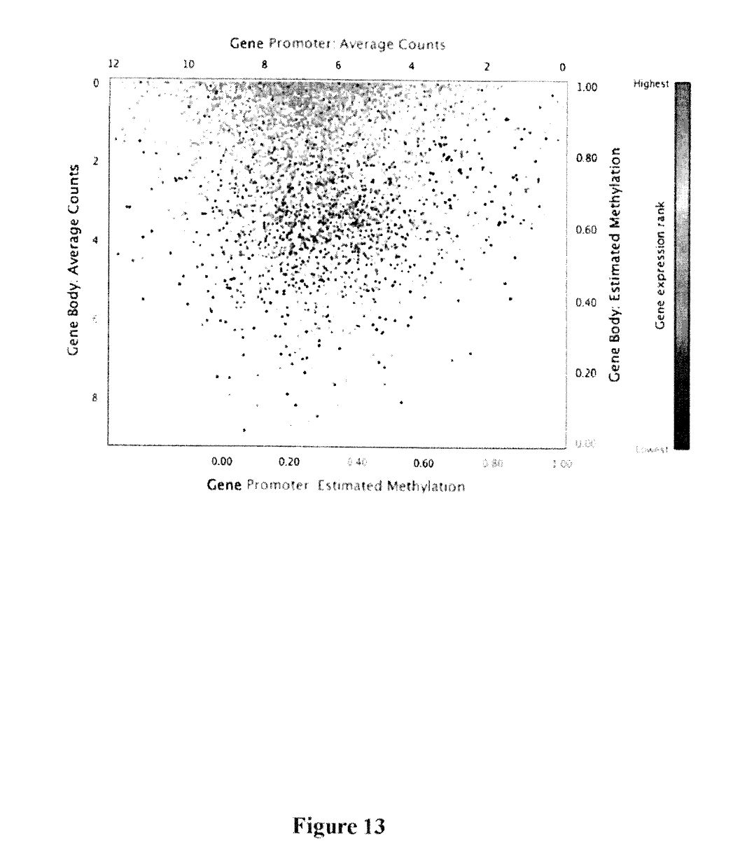

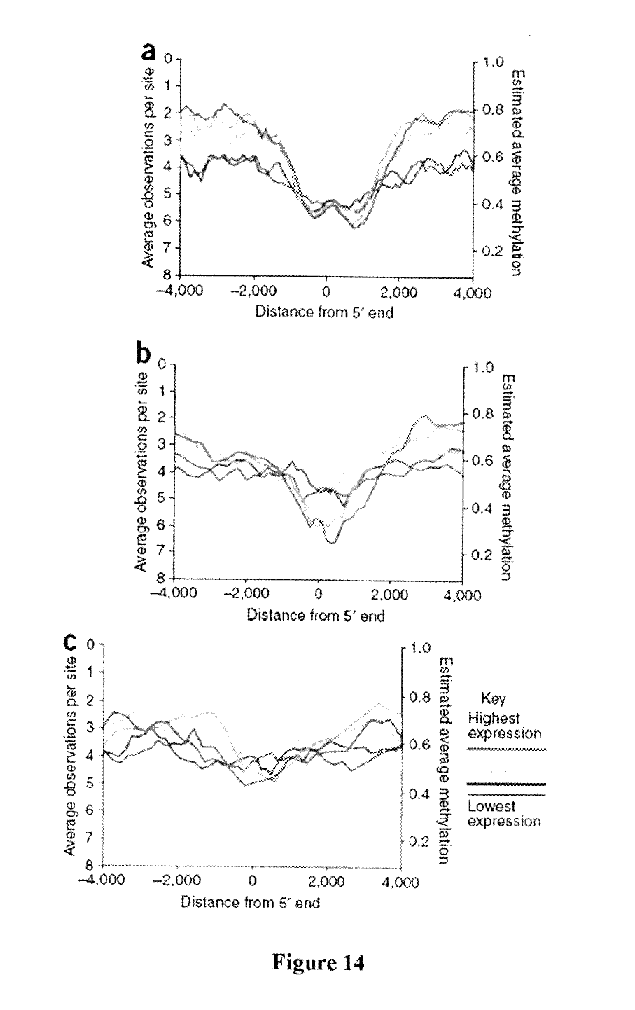

Based the analyses presented herein (mainly FIG. 2), it was decided that there were two methylation measurements that might be relevant to classifying genes according to epigenetic state: promoter methylation and gene body methylation. Based on FIG. 2C, the promoter region was defined as spanning −400 base pairs to +1000 base pairs relative to transcription start. Based on FIGS. 2C and 2D, the gene body region was defined as the region between +3000 base pairs relative to transcription start and the end of the gene. For each gene, all the data points from the promoter and gene body regions were gathered and, using the genes that had at least ten data points in each region, each gene was plotted according to the average counts for each region (FIG. 13, x axis=gene promoter, y axis=gene body, color=gene expression rank). Because a larger difference was observed, on average, in promoter methylation relative to gene expression for data points arising from outside CpG islands, the same promoter vs. methylation graph was generated using only data points from outside CpG islands (FIG. 3A). For this plot a minimum of five data points were required for each region.

To create a histogram of average gene body methylations individual genes, the same gene body methylation averages were used, but the set was restricted to genes with 50 or more data points within that region (FIG. 3B).

What is claimed is:

1. A method for determining a methylated cytosine profile of a target nucleic acid sequence comprising the steps of:
providing a sample of nucleic acid sequences;
contacting the sample with a chemical agent that converts unmethylated cytosine residues in the nucleic acid sequences to uracil residues;
contacting the sample with a plurality of nucleic acid probes, wherein the probes are designed to hybridize randomly along a target nucleic acid sequence;
allowing hybridization of the plurality of nucleic acid probes to the target nucleic acid sequence;
forming a plurality of circular nucleic acid sequences, each of the circular sequences comprising a nucleic acid probe sequence and a target nucleic acid sequence;
amplifying the plurality of circular nucleic acid sequences to form a plurality of amplified target nucleic acid sequences; and
sequencing the amplified target nucleic acid sequences.

2. The method of claim 1, wherein the chemical agent is bisulfate.

3. The method of claim 1, wherein the probes are designed to hybridize to promoter regions along a target nucleic acid sequence.

4. The method of claim 1, wherein amplification primers hybridize to nucleic acid probe sequences during the step of amplifying.

5. The method of claim 1, wherein the nucleic acid probes are padlock probes.

6. The method of claim 1, wherein the target nucleic acid sequence is genomic DNA.

7. The method of claim 6, wherein the genomic DNA is whole genome DNA.

8. The method of claim 1, wherein the target nucleic acid sequence is a gene.

9. The method of claim 1, wherein the target nucleic acid sequence is a promoter region.

10. A method for determining a methylated cytosine profile of a target nucleic acid sequence comprising the steps of:
providing a sample of nucleic acid sequences;
cleaving the nucleic acid sequences in a methylation-dependent manner to generate a plurality of cleaved target nucleic acid sequences;
ligating first adapter sequence tags to the 5′ ends of cleaved target nucleic acid sequences and second adapter sequence tags to the 3′ ends of the cleaved target nucleic acid sequences;
amplifying the cleaved target nucleic acid sequences having first and second adapter sequence tags ligated thereto; and
sequencing the amplified, cleaved target nucleic acid sequences.

11. The method of claim 10, wherein the step of cleaving the nucleic acid sequences in a methylation-dependent manner comprises contacting the nucleic acid sequences with a methyl sensitive restriction enzyme to cleave unmethylated CpG dinucleotide sequences.

12. The method of claim 10, wherein amplification primers hybridize to the first or the second adapter sequence tags during the step of amplifying.

13. The method of claim 10, wherein the target nucleic acid sequence is genomic DNA.

14. The method of claim 13, wherein the genomic DNA is whole genome DNA.

15. The method of claim 10, wherein the target nucleic acid sequence is a gene.

16. The method of claim 10, wherein the target nucleic acid sequence is a promoter region.

17. The method of claim 10, further comprising the step of comparing the methylated cytosine profile of the target nucleic acid sequence to a methylated cytosine profile of a control library.

18. The method of claim 17, wherein the control library is generated by contacting a target nucleic acid sequence with a methylation-insensitive enzyme.

19. The method of claim 18, wherein the methylation-insensitive enzyme is MspI.

20. A method for determining a complementary methylated cytosine library of a target nucleic acid sequence comprising the steps of:
providing a sample of nucleic acid sequences;
cleaving the nucleic acid sequences in a methylation-dependent manner to generate a plurality of cleaved target nucleic acid sequences;
blocking the ends of the cleaved target nucleic acid sequences to prevent the cleaved target nucleic acid sequences from contributing to library construction; and
contacting the blocked, cleaved target nucleic acid sequences with a methylation-insensitive enzyme to create a complementary methylated cytosine library that comprises a plurality of nucleic acid sequences that were not cleaved in a methylation-dependent manner.

21. The method of claim 20, wherein the blocking step comprises dephosphorylating the 5′ ends of the cleaved target nucleic acid sequences.
